(19) United States
(12) Patent Application Publication
(10) Pub. No.: US 2025/0172568 A1
(43) Pub. Date: May 29, 2025

(54) AFUCOSYLATION OF HLA-SPECIFIC IGG1 AS A POTENTIAL PREDICTOR OF ANTIBODY PATHOGENICITY IN KIDNEY TRANSPLANTATION

(71) Applicants: Margaret ACKERMAN, Hanover, NH (US); Sweta SHRESTHA, Hanover, NH (US); Pranay BHARADWAJ, Danbury, CT (US); Manfred WUHRER, Voorschoten (NL); Arnaud MARCHANT, Braine l’Alleud (BE); Alain LE MOINE, Brussels (BE); Tamas PONGRACZ, Leiden (NL); Noortje DE HAAN, Copenhagen (DK); TRUSTEES OF DARTMOUTH COLLEGE, Hanover, NH (US); UNIVERSITÉ LIBRE DE BRUXELLES, Brussels (BE); LEIDEN UNIVERSITY MEDICAL CENTRE, Leiden (NL)

(72) Inventors: Margaret ACKERMAN, Hanover, NH (US); Sweta SHRESTHA, Hanover, NH (US); Pranay BHARADWAJ, Danbury, CT (US); Manfred WUHRER, Voorschoten (NL); Arnaud MARCHANT, Braine l’Alleud (BE); Alain LE MOINE, Brussels (BE); Tamas PONGRACZ, Leiden (NL); Noortje DE HAAN, Copenhagen (DK)

(21) Appl. No.: 18/841,266

(22) PCT Filed: Feb. 24, 2023
(86) PCT No.: PCT/US2023/063205

§ 371 Date: Aug. 23, 2024

Related U.S. Application Data
(60) Provisional application No. 63/313,953, filed on Feb. 25, 2022.

(51) Int. Cl. G01N 33/68; C12Q 1/37; G01N 33/543; G01N 33/564

(52) U.S. Cl. CPC G01N 33/6854; C12Q 1/37; G01N 33/54326; G01N 33/564; G01N 2333/70535; G01N 2333/70539; G01N 2333/976; G01N 2440/38; G01N 2800/245; G01N 2800/347; G01N 2800/50

(57) ABSTRACT

The present disclosure relates to methods for predicting and improving the outcome of organ transplantation based on features of donor-specific antibodies (DSAs) present in the transplant candidate or recipient. Anti-DSA features include FcγRIIA binding and Fc glycosylation.

### CROSS REFERENCE TO RELATED APPLICATIONS

This patent application claims priority to U.S. Provisional Patent Application No. 63/313,953, filed on Feb. 25, 2022, the entire contents of which are fully incorporated herein by reference.

### SEQUENCE LISTING

The computer-readable Sequence Listing submitted on Feb. 24, 2023 and identified as follows: 15,036 bytes ST.26 XML document file named “029511-8110 Sequence Listing.xml,” created Feb. 24, 2023, is incorporated herein by reference in its entirety.

### TECHNICAL FIELD

The present invention relates to methods for predicting and improving the outcome of organ transplantation based on features of donor-specific antibodies (DSAs) present in the transplant candidate or recipient. Anti-DSA features include FcγRIIIA binding and Fc glycosylation.

### BACKGROUND

Antibody-mediated rejection (AMR), encompassing allograft rejection caused primarily by antibodies directed against donor-specific human leukocyte antigen (HLA) molecules, is the leading cause of solid organ transplant rejection and long-term graft loss. AMR is characterized by histological manifestations of endothelial cell injury, mononuclear cell infiltration, and complement-dependent tissue damage. Additionally, the presence of circulating donor-specific antibodies (DSAs) poses challenges for transplant recipients by limiting access to available organs, prolonging wait time, and, in some cases, excluding the candidates from a possible transplantation altogether. Solid phase assays using purified HLA antigens (LUMINEX® single bead antigen assays) have significantly improved stratification and categorization of transplant candidates because they can detect very low levels of DSAs due to their high sensitivity. While this technique has been used to update organ allocation and desensitization protocols, it has led to minimal benefits to improvement in rejection treatment as the presence and levels of DSAs are not a reliable predictor of transplant outcome. While graft survival is clearly poorer for individuals sensitized against donor organ antigens, several studies have shown that not all DSAs carry the same risk of allograft rejection, as they have been associated with a wide spectrum of effects ranging from a complete absence of graft injury to the most severe form of AMR. Similarly, appearance of de novo DSAs implies the risk of graft deterioration but provides little to no information on their actual pathogenic activities.

Collectively, these observations point to the importance of factors other than the magnitude of the DSA response as important drivers of pathology. Clinical practice is challenged by the lack of strong relationships between antibody characteristics and patient outcomes. Given the profound disproportion between available organs and the number of patients waiting for a transplant, the US Food and Drug Administration (FDA) recently identified AMR and desensitization as two important areas in transplantation for which no drugs have been specifically approved. Techniques that better define DSA properties may be required to first advance mechanistic understanding of AMR and to secondly improve transplant recipient outcomes.

To this end, the antibody effector functions that may be responsible for AMR are influenced not solely by titer, but by affinity, antigen availability and epitope, and antibody isotype, subclass, and glycosylation. In other disease settings, systematic tools for surveillance of this spectrum of serum antibody features and their associated effector functions have identified reliable associations and begun to support robust predictions of disease outcomes.

While prior research on the role of antibodies in transplant rejection was predominantly focused on estimation and retrospective correlation of titer to better predict transplant outcomes, recent studies have begun to interrogate other important factors governing antibody functionality, such as subclass distribution and complement fixing ability, and the evolution of these features with the progression of disease.

### SUMMARY OF THE INVENTION

The present disclosure relates to methods for predicting and improving the outcome of organ transplantation based on features of donor-specific antibodies (DSAs) present in the transplant candidate or recipient. Anti-DSA features include FcγRIII binding and Fc glycosylation. In one aspect, the FcγRIII binding and/or glycosylation feature of DSAs are used to predict and improve the outcome of organ transplantation.

Antibody-mediated rejection (AMR) is the leading cause of graft failure. While donor-specific antibodies (DSAs) are associated with a higher risk of AMR, not all patients with DSAs develop rejection, suggesting that the characteristics of alloantibodies that determine their pathogenicity remain undefined. Using human leukocyte antigen (HLA)-A2-specific antibodies as a model, systems serology tools were applied to investigate qualitative features of immunoglobulin G (IgG) alloantibodies including Fc-glycosylation patterns and FcγR-binding properties. Levels of afucosylated anti-A2 antibodies were elevated in all seropositive patients and were significantly higher in AMR patients, suggesting potential cytotoxicity via FcγRIII-mediated mechanisms. Afucosylation of both glycoengineered monoclonal and naturally glycovariant polyclonal serum IgG specific to HLA-A2 exhibited potentiated binding to, slower dissociation from, and enhanced signaling through FcγRIII, a receptor widely expressed on innate effector cells. Collectively, these results suggest that afucosylated DSA may be a biomarker of AMR and could contribute to pathogenesis.

The present disclosure reports on the development and application of novel assays to characterize phenotypic and functional aspects of HLA-specific antibodies. Among a group of individuals with antibodies against HLA-A2, we observe altered Fc glycosylation profiles compared with total serum immunoglobulin G (IgG)—most notably, enrichment of afucosylated IgG1 antibodies, which are widely associated with potentiated antibody-dependent cellular cytotoxicity (ADCC). For both glycoengineered monoclonal as well as polyclonal, human serum-derived HLA-A2-specific antibodies, binding to, and signaling via FcγRIIIa, the receptor expressed on NK and other innate immune cells and responsible for mediating ADCC, were negatively associated with fucosylation. Collectively, this work establishes the potential importance of DSA Fc glycosylation in influencing the ADCC activity of DSA and, in turn, their ability to contribute to graft rejection.

Thus, in one aspect, the present disclosure contemplates methods for predicting risk of AMR in a candidate for or recipient of a solid organ allograft.

In another aspect, the present disclosure contemplates methods for screening, identifying, stratifying, categorizing, monitoring, and/or treating a candidate for or a recipient of a solid organ allograft.

In certain embodiments of any aspect disclosed herein, the methods comprise determining or having determined a feature of a DSA obtained from a sample from the candidate or recipient. In some such embodiments, the feature of the DSA is selected from the group consisting of IgG subclass, Fc domain glycosylation, FcγR binding, and combinations thereof. In some such embodiments, the feature of the DSA is an Fc feature, such as Fc domain glycosylation and/or FcγR binding. In some such embodiments, the DSA is an anti-HLA antibody, such as an anti-HLA-A2 antibody and in particular an anti-HLA-A2 IgG antibody.

In certain embodiments of any aspect disclosed herein, low DSA fucosylation is a marker of AMR risk in the candidate or recipient. In some such embodiments, the solid organ is a kidney. In some such embodiments, the DSA is an anti-HLA antibody, such as an anti-HLA-A2 antibody and in particular an anti-HLA-A2 IgG antibody. Thus, in one embodiment, low HLA-A2 IgG1 fucosylation is a marker of AMR risk in kidney transplant candidates and/or recipients.

### BRIEF DESCRIPTION OF DRAWINGS

For a better understanding of the invention, reference may be made to embodiments shown in the following drawings. The components in the drawings are not necessarily to scale and related elements may be omitted, or in some instances proportions may have been exaggerated, so as to emphasize and clearly illustrate the novel features described herein. In addition, system components can be variously arranged, as known in the art.

FIGS. 1A-1B. Subclass distribution of HLA-specific antibodies. Characterization of HLA-specific antibodies binding to HLA-A2 (1A) and HLA-A1(1B) antigens across IgG subclasses in HLA-A2-positive (hollow black circles; n=30-32) and control (hollow gray circles; n=18) individuals. HLA-A2-specific BB7.2 (square) and HIV-specific VRC01 (triangle) mAb subclass controls for HLA-A2 reactivity are shown for each subclass: IgG1 (light blue), IgG2 (orange), IgG3 (dark blue), and IgG4 (red). Buffer only blank (cross) and pooled IVIG (diamond) are shown in brown and purple, respectively. Serum samples were tested at a 1:100 dilution. Data shown are representative of two technical replicates. Solid red lines indicate group median. Differences between groups were evaluated using ordinary two-way ANOVA adjusted for multiple comparisons using Bonferroni's test (** p<0.01, *** p<0.001, and **** p<0.0001, respectively).

FIGS. 2A-2D. Fc glycosylation of HLA-A2-specific antibodies. Representative extracted ion chromatograms and mass spectra (2A and 2B) illustrating the observed variability between HLA-A2-specific (2A) and total (2B) IgG1 Fc glycosylation patterns of the same patient. (2C) Volcano plot displaying the logio-fold change (x-axis) and -logio p value (y-axis) of individual IgG1 (blue circle) and IgG2/3 (maroon square) glycoforms between total and HLA-A2-specific IgG1 and IgG2/3, respectively. (2D) Violin plots showing the relative prevalence of glycans on bulk and HLA-A2-specific IgG1 (top) and IgG2/3 (bottom), respectively. Statistical analysis was performed using a paired two-tailed Student's t test (** p<0.01, *** p<0.001, and **** p<0.0001, respectively).

FIGS. 3A-3D. Impact of HLA-A2-specific mAb fucosylation on FcγRIIIa binding, signaling, and cytotoxic activity per subclass. (3A) FcγRIIIa signaling in a reporter cell line assay with unmodified and afucosylated HLA-A2 mAbs of varying subclasses. (3B) Death (percentage of cytotoxicity) of HLA-A2+target cells following coculture with NK cells in the presence or absence of antibodies of varying specificity, subclass, and fucosylation status. Connecting lines indicate curve fit models. Error bars indicate mean and SD of duplicates. Dotted horizontal line represents ADCC activity in the absence of antibody. (3C) Schematic figure of the BLI experiment to define FcγRIIIa off-rates from HLA-A2-specific antibodies. (3D) FcγRIIIa V158 association with and dissociation from unmodified and afucosylated HLA-A2 mAbs. Dissociation rate (kd) values are shown in inset.

FIGS. 4A-4D. Associations of serum-derived HLA-A2-specific antibody fucosylation with FcγRIIIa binding and signaling. (4A) Spearman's correlation (Rs) between IgG1 fucosylation and FcγRIIIa dissociation rate (n=13). (4B) FcγRIIIa binding characterization in high (n=8), medium (n=8), and low (n=8) fucose samples and negative controls (n=18). Serum samples were tested at a 1:500 serum dilution. Statistical analysis was performed using ordinary one-way ANOVA adjusted for multiple comparisons using Tukey's test. Solid lines indicate group median. Data shown are representative of two technical replicates. (4C and 4D) Spearman's correlations between IgG1 fucosylation (n=24) (4C) and FcγRIIIa signaling (n=31) (4D) with FcγRIIIa binding (left) and HLA-A2-specific IgG levels (mean fluorescence intensity [MFI]) (right). Patients with AMR (red), patients without AMR (green), and patients with no AMR information (black) are indicated in color.

FIGS. 5A-5C. HLA-A2-specific IgG1 afucosylation is associated with AMR. (5A) Violin plot showing relative prevalence of fucose on HLA-A2-specific IgG1 in individuals with AMR (n=10) and without AMR (n=9). A Mann-Whitney U test was used to compare the two groups. (5B) Receiver operating characteristic (ROC) curve and area under the curve (AUC) depicting performance of AMR status classification across increasing IgG1 fucose prevalence thresholds. (5C) Number of subjects in which HLA-A2-specific IgG1 comprise a DSA plotted by fucose content as tertiles (low, medium, high) and AMR status. Statistical significance defined by Fisher's exact test.

FIGS. 6A-6B. mAb controls for subclassing assay. Dose response profiles of HLA-A2 (positive control) and VRCO1 (negative control) mAbs to HLA-A2 (6A) and HLA-A1 (6B) antigens across IgG subclasses. HLA-A2-specific BB7.2 (square) and HIV-specific VRCO1 (triangle) mAb subclass controls are shown for each subclass; IgG1 (light blue), IgG2 (orange), IgG3 (dark blue) and IgG4 (red). Baseline (buffer only control) signal is indicated by dotted lines.

FIG. 7. Relationship between HLA-A2 specific IgG signal and signal reported from the clinical testing. Correlation between HLA-A2 specific IgG MFI and values reported from the clinical testing (n=30). Spearman rank correlation (RS) and corresponding p-value are shown in inset.

FIGS. 8A-8G. Antigen specific antibody purification. (8A) Schematic of affinity purification and Fc-glycosylation analysis of HLA-A2 specific antibodies. HLA-A2 specific antibodies were purified using HLA-A2 antigen coated magnetic beads. Diluted serum was incubated with magnetic beads to allow binding of HLA-A2-specific antibodies. Beads were washed and then the bound antibodies were eluted. Fc-glycosylation analysis of the eluted HLA-A2 specific antibodies was performed by liquid chromatography-mass spectrometry on the glycopeptide level following tryptic digestion. (8B-8D) Proof of concept for antigen specific antibody purification. Reactivity of IVIG spiked with 0.1% (8B), 1.0% (8C) and 5.0% (8D) murine HLA-A2 mAb to HLA-A2 (top), HLA-A1 (middle) and HSV-gD (bottom) antigens. Load and Flow-through are shown in black and grey, respectively. Reactivity of Eluate for HLA-A2, HLA-A1 and HSV-gD are shown in blue, green, and red, respectively. (8E-8G) Antigen specific antibody purification profiles. Reactivity of purified HLA-A2 specific antibodies to HLA-A2 (8E), HLA-A1 (8F) and HSV-gD (8G) antigens from HLA-A2 positive (n=29) individuals. Signal for each antigen specificity in plotted on the y-axis and signal from total IgG on the x-axis. Profiles of the serum antibodies before purification (loads) are shown in black.

FIGS. 9A-9B. Antibody-Receptor dissociation kinetics measurement by Biolayer Interferometry (BLI). (9A) Schematic of the Antibody-Receptor dissociation kinetics measurement by Biolayer Interferometry (BLI). Streptavidin tips were first coated with biotinylated HLA-A2 antigens. After establishing a signal baseline, the tips were loaded with HLA-A2-specific antibodies, dipped into the buffer for baseline, and then finally dipped into a solution of recombinantly expressed FcγRIIIa V158 monomers. Following receptor association, tips were dipped into a buffer, which allowed receptor dissociation. The dissociation rate of FcγR was defined relatively to a reference tip which was not dipped into the receptor. (9B) FcγRI binding characterization of unmodified and afucosylated HLA-A2 IgG1 and IgG3 mAbs. FcγRI association with and dissociation from unmodified and afucosylated HLA-A2 mAbs. Dissociation rates (k_{d}) are shown in inset.

FIGS. 10A-10F. HLA-A2 specific antibodies characteristics in patient serum samples. FcγRIIIA V158 signaling characterization of polyclonal serum HLA-A2-specific antibodies. (10A) FcγRIIIa signaling in a reporter cell line assay with high (n=7-8), medium (n=7) and low (n=8) IgG1 fucose content, and controls (n=18) in three independent assay replicates. ADCC assay performed with direct antigen (left and middle), and neutravidin-antigen (right) coated on the plate. Statistical analysis was performed using Ordinary one-way ANOVA adjusted for multiple comparisons using Tukey's test (*p<0.05). (10B) Correlations between FcγRIIIa signaling to FcγRIIIa binding (middle panel) and HLA-A2 specific IgG MFI (lower panel) (n=30-31) across Fc signaling assay replicates. Spearman rank correlations (RS) are shown in inset. (10C-10F). HLA-A2-specific antibody characteristics in individuals with and without AMR. Violin plots showing HLA-A2-specific IgG MFI in individuals with AMR (n=13) versus no AMR (n=13) (10C), FcγRIIIa binding characterization in individuals with AMR (n=13) versus no AMR (n=13) (10D), FcγRIIIa signaling characterization in individuals with AMR (n=12) versus no AMR (n=13) (10E) and FcγRIIIa dissociation rate in individuals with AMR (n=8) versus no AMR (n=2) (10F). Patients with and without AMR are shown in red and green, respectively. Statistical analysis was performed using a Mann-Whitney U test.

FIG. 11. HLA-A2-specific monoclonal antibody gene sequences. FIG. 11 shows exemplary gene sequences for light chain (SEQ ID NO: 1), IgG1 heavy chain (SEQ ID NO: 2), IgG2 heavy chain (SEQ ID NO: 3), IgG3 heavy chain (SEQ ID NO: 4), and IgG4 heavy chain (SEQ ID NO: 5).

### DETAILED DESCRIPTION

This detailed description is intended only to acquaint others skilled in the art with the present invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

### A. DEFINITIONS

As used in the specification and the appended claims, unless specified to the contrary, the following terms have the meaning indicated:

The term “allograft” refers to an organ or tissue that is transplanted from one individual to another individual of the same species with a different genotype. An allograft can be contrasted with an autograft, which refers to a graft from one point to another of the same individual's body. Allografts are provided by donors and can be from a living or deceased (e.g., cadaveric) source. Preferably, the allograft is a solid organ, such as kidney, liver, intestines, heart, lung and pancreas, or a portion thereof. Alternatively, the allograft can be a tissue, such as bone, tendon, or skin.

The term “antibody” refers to a glycoprotein comprising at least two heavy (H) chains and two light (L) chains inter-connected by disulfide bonds, and any molecule comprising an antigen-binding portion of such glycoprotein. Exemplary molecules encompassed by the term “antibody” include single chain antibodies (e.g., single chain Fv (scFv)), Fab and Fab′ fragments. In the context of a prophylactic or therapeutic antibody, such antibody can be, for example, a monoclonal antibody or a polyclonal antibody.

An IgG antibody typically comprises a pair of heavy chains, each heavy chain comprised of a heavy chain variable region (abbreviated herein as VH) and a heavy chain constant region and a pair of light chains, each light chain comprised of a light chain variable region (abbreviated herein as VL) and a light chain constant region. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant region of the heavy chain can also be further subdivided into multiple domains such as CH1, CH2, and CH3.

The term “Fc” or “Fc domain” or “Fc region” refers to the non-antigen binding portion or “fragment crystallizable region” of an immunoglobulin or antibody, or fragment thereof. The Fc domain may mediate the binding of the immunoglobulin to host tissues or factors, including various cells of the immune system (e.g., effector cells) and the first component (Clq) of the classical complement system. The Fc domain may mediate effector functions, such as binding to FcR and inducing immune responses. An IgG Fc domain typically comprises two CH2 and CH3 domains.

The term “Fc receptor” or “FcR” refers to a binding partner for an Fc domain. The FcR may be a soluble Fc binding fragment but typically is a cell surface receptor.

The terms “treat”, “treating” and “treatment” refer to both therapeutic and preventative or prophylactic measures to alleviate or abrogate a condition, disorder, or disease and/or the attendant symptoms thereof.

In this application, the use of the disjunctive is intended to include the conjunctive. The use of definite or indefinite articles is not intended to indicate cardinality. In particular, a reference to “the” object or “a” and “an” object is intended to denote also one of a possible plurality of such objects. Further, the conjunction “or” may be used to convey features that are simultaneously present instead of mutually exclusive alternatives. In other words, the conjunction “or” should be understood to include “and/or”. The terms “includes,” “including,” and “include” are inclusive and have the same scope as “comprises,” “comprising,” and “comprise” respectively.

### B. EXEMPLARY METHODS

In one aspect, the present disclosure provides a method for assessing if a candidate for or a recipient of a solid organ allograft is at risk of developing antibody-mediated rejection (AMR). The method comprises determining or having determined a feature of a donor-specific antibody (DSA) in a sample obtained from the candidate or recipient.

In certain embodiments, the feature of the DSA is selected from the group consisting of IgG subclass, Fc domain glycosylation, FcγR binding properties, and combinations thereof. In some such embodiments, the feature of the DSA is an Fc feature, such as Fc domain glycosylation and/or FcγR binding properties.

In certain embodiments, the method further comprises identifying the candidate or the recipient as having a high risk of AMR based on the DSA having a low level of fucosylation and/or exhibiting slow dissociation from FcγRIIIa, enhanced FcγRIIIa signaling, and/or ADCC activity.

In certain embodiments, the DSA is HLA-A2-specific IgG1 and low fucosylation, high FcγRIIIa binding, high signaling activity, and/or slow dissociation from FcγRIIIa is indicative of a high risk for AMR.

In certain embodiments, the method further comprises (i) allocating a solid organ allograft for the candidate; (ii) selecting the candidate for transplant surgery; and/or (iii) selecting the candidate for treatment with an immunosuppressive therapy based on the feature(s) of the DSA. In some such embodiments, the immunosuppressive therapy is an immunosuppressive induction therapy.

In certain embodiments, the method further comprises selecting the recipient for treatment with an immunosuppressive therapy based on the feature(s) of the DSA. In some such embodiments, the immunosuppressive therapy is an immunosuppressive maintenance therapy.

In certain embodiments, the method further comprises treating the candidate or the recipient with an immunosuppressive therapy if the candidate or recipient has an increased risk of AMR. In some such embodiments, the treatment step comprises administering one or more immunosuppressants to the candidate or recipient.

In one aspect, the present disclosure provides a method for treating a patient in need of a solid organ transplant. The method comprises transplanting a solid organ from a donor to the patient, wherein prior to said transplanting a sample from said patient has been assessed to determine a feature of a donor-specific antibody (DSA).

In certain embodiments, the feature of the DSA is selected from the group consisting of IgG subclass, Fc domain glycosylation, FcγR binding properties, and combinations thereof. In some such embodiments, the feature of the DSA is an Fc feature, such as Fc domain glycosylation and/or FcγR binding properties.

In certain embodiments, the method further comprises treating the patient with an immunosuppressive therapy if the patient has been identified as having an increased risk of

AMR. In some such embodiments, the treatment step comprises administering one or more immunosuppressants to the patient. In some such embodiments, the immunosuppressive therapy is an immunosuppressive maintenance therapy.

The transplant procedure may take place at a specially-designated treatment facility or transplant center.

In one aspect, the present disclosure provides a method for monitoring and treating a recipient of a solid organ allograft. The method comprises determining or having determined a feature of a donor-specific antibody (DSA) in a sample obtained from the recipient.

In certain embodiments, the feature of the DSA is selected from the group consisting of IgG subclass, Fc domain glycosylation, FcγR binding properties, and combinations thereof. In some such embodiments, the feature of the DSA is an Fc feature, such as Fc domain glycosylation and/or FcγR binding properties.

In certain embodiments, a low level of DSA fucosylation indicates that the recipient has a high risk of AMR.

In certain embodiments, a DSA exhibiting slow dissociation from FcγRIIIa, enhanced FcγRIIIa signaling, and/or ADCC activity indicates that the recipient has a high risk of AMR.

In certain embodiments, the method further comprises (1) increasing the amount and/or frequency of a maintenance immunosuppressive therapy if the feature of the DSA indicates that the recipient has an high risk of developing AMR; (2) maintaining the amount and/or frequency of a maintenance immunosuppressive therapy if the feature of the DSA indicates that the recipient does not have a high risk or has a low risk of developing AMR; or (3) decreasing the amount and/or frequency of a maintenance immunosuppressive therapy if the feature of the DSA indicates that the recipient does not have a high risk or has a low risk of developing AMR.

In one aspect, the present disclosure provides a method for identifying and treating a renal allograft candidate or recipient at risk of developing antibody-mediated rejection (AMR). The method comprises (a) determining or having determined a feature of a donor-specific antibody (DSA) in a sample obtained from the candidate or recipient; (b) quantitatively or qualitatively comparing the feature of the DSA with a reference value; (c) identifying the renal allograft candidate or recipient as being at risk for allograft rejection based the comparison in step (b), and (d) providing a therapeutic intervention to the candidate or recipient identified in step (c) as being at risk of allograft rejection.

In certain embodiments, the feature of the DSA is selected from the group consisting of IgG subclass, Fc domain glycosylation, FcγR binding properties, and combinations thereof. In some such embodiments, the feature of the DSA is an Fc feature, such as Fc domain glycosylation and/or FcγR binding properties.

In certain embodiments, the reference value is obtained from a corresponding feature previously determined from samples obtained from renal allograft recipients who did not have clinically defined AMR or a group of otherwise healthy individuals.

In certain embodiments, the therapeutic intervention comprises administration of one or more immunosuppressants.

In certain embodiments, the therapeutic intervention comprises induction immunosuppressive therapy. In certain embodiments, the therapeutic intervention comprises maintenance immunosuppressive therapy.

In one aspect, the present disclosure provides a method for identifying and treating a renal allograft candidate at risk of developing antibody-mediated rejection (AMR). The method comprises (a) determining or having determined a feature of a donor-specific antibody (DSA) in a sample obtained from the candidate; (b) quantitatively or qualitatively comparing the feature of the DSA with a reference value; (c) identifying the renal allograft candidate as being at risk for allograft rejection based the comparison in step (b), and (d) administering induction immunosuppressive therapy to the candidate identified in step (c) as being at risk of allograft rejection.

In certain embodiments, the feature of the DSA is selected from the group consisting of IgG subclass, Fc domain glycosylation, FcγR binding properties, and combinations thereof. In some such embodiments, the feature of the DSA is an Fc feature, such as Fc domain glycosylation and/or FcγR binding properties.

In certain embodiments, the reference value is obtained from a corresponding feature previously determined from samples obtained from renal allograft recipients who did not have clinically defined AMR or a group of otherwise healthy individuals.

In one aspect, the present disclosure provides a method for identifying and treating a renal allograft recipient at risk of developing antibody-mediated rejection (AMR). The method comprises (a) determining or having determined a feature of a donor-specific antibody (DSA) in a sample obtained from the recipient; (b) quantitatively or qualitatively comparing the feature of the DSA with a reference value; (c) identifying the renal allograft recipient as being at risk for allograft rejection based the comparison in step (b), and (d) administering maintenance immunosuppressive therapy to the recipient identified in step (c) as being at risk of allograft rejection.

In certain embodiments, the feature of the DSA is selected from the group consisting of IgG subclass, Fc domain glycosylation, FcγR binding properties, and combinations thereof. In some such embodiments, the feature of the DSA is an Fc feature, such as Fc domain glycosylation and/or FcγR binding properties.

In certain embodiments, the reference value is obtained from a corresponding feature previously determined from samples obtained from renal allograft recipients who did not have clinically defined AMR or a group of otherwise healthy individuals.

In one aspect, the present disclosure provides an in vitro method for determining the likelihood of occurrence of antibody-mediated rejection (AMR) against a renal allograft in a transplant candidate or recipient. The method comprises the steps of: (a) obtaining a sample containing donor-specific antibodies (DSAs) from the candidate or recipient; (b) affinity purifying DSAs from the sample using a donor-specific antigen attached to a solid support; (c) enzymatically digesting the affinity purified DSAs to obtain Fc glycopeptides; (d) determining glycosylation profiles of the Fc glycopeptides obtained in step (c); (e) comparing the glycosylation profile obtained in step (d) with a reference value; and (f) determining the likelihood of occurrence of AMR against a renal allograft in the candidate or recipient based on the comparison of step (e).

In certain embodiments, the sample is a blood sample. In certain embodiments, the sample is a serum sample.

In certain embodiments, the solid support is a bead. In some such embodiments, the bead is a magnetic bead. In some such embodiments, the bead is a streptavidin coated bead. For example, the solid support can be a magnetic streptavidin coated bead.

In certain embodiments, the donor-specific antigen is HLA. In some such embodiments, the donor-specific antigen is HLA-A2.

In certain embodiments, the donor-specific antigen comprises a label that allows for attachment to the solid support. In some such embodiments, the donor-specific antigen is biotinylated.

In certain embodiments, the enzymatic digestion comprises tryptic digestion. In certain embodiments, the enzymatic digestion employs trypsin.

In certain embodiments, the Fc glycosylation profile is a Fc fucosylation profile.

In certain embodiments, the reference value is obtained from an Fc glycosylation profile previously determined from samples obtained from renal allograft recipients who did not have clinically defined AMR or a group of otherwise healthy individuals.

In certain embodiments, a low level of DSA fucosylation indicates a high likelihood of occurrence AMR.

In one aspect, the present disclosure provides a method for treating antibody-mediated rejection (AMR) in a candidate for or a recipient of a solid organ allograft, particularly a renal allograft. The method comprises the steps of (a) determining the likelihood of occurrence of AMR by (1) determining or having determined a feature of a donor-specific antibody (DSA) in a sample obtained from the candidate or recipient; (2) quantitatively or qualitatively comparing the feature of the DSA determined in step (a)(1) with a reference value; (3) determining the likelihood of occurrence of AMR based on the comparison of step (a)(2); (b) selecting the candidate or recipient when the candidate or recipient has been determined as being likely to develop AMR at step (a); and (c) treating the candidate or recipient selected at step (b) with an immunosuppressive therapy.

In certain embodiments of any aspect disclosed herein, the DSA is an anti-HLA antibody. In some such embodiments, the anti-HLA antibody is an anti-HLA-A2 antibody. In some such embodiments, the anti-HLA-A2 antibody is an anti-HLA-A2 IgG1 antibody.

In certain embodiments of any aspect disclosed herein, the solid organ is a kidney.

In certain embodiments of any aspect disclosed herein, the biological sample is a blood sample, such as a serum sample.

In certain embodiments of any aspect disclosed herein, Fc domain glycosylation and/or FcγR binding properties are determined with reference to with a control group, such as a group of allograft recipients who did not have clinically defined AMR or a group of otherwise healthy individuals. For example, the level of fucosylation may be determined by comparing the fucosylation of the subject DSA to fucosylation of an antibody, preferably a DSA, obtained from allograft recipient(s) who did not have clinically defined AMR.

In certain embodiments of any aspect disclosed herein, Fc domain glycosylation and/or FcγR binding properties are determined with reference to one or more predetermined cutoff values. The predetermined cutoff value(s) may be established using data obtained from a control group. The predetermined cutoff value(s) may divide into two groups (e.g., low, high), tertiles (e.g., low, medium, high), quartiles, etc.

In certain embodiments of any aspect disclosed herein, a low level of fucosylation of the DSA (e.g., anti-HLA antibody) indicates an increased risk for developing an AMR. In some such embodiments, the low levels of fucosylation are relative to a predetermined cutoff value and/or the level of fucosylation of DSA from otherwise healthy individuals or allograft recipients who did not have AMR.

In certain embodiments of any aspect disclosed herein, the immunosuppressive therapy comprises administration one or more immunosuppressants. Exemplary immunosuppressants include calcineurin inhibitors, antiproliferative agents, glucocorticoids, mammalian target of rapamycin (mTOR) inhibitors, and antibodies.

In certain embodiments of any aspect disclosed herein, the immunosuppressant is a calcineurin inhibitor. Oral and injectable calcineurin inhibitors are used for both the induction and maintenance of immunosuppression. Calcineurin inhibitors include, for example, tacrolimus and cyclosporine.

An exemplary calcineurin inhibitor is tacrolimus, previously known as FK506. Tacrolimus is a macrolide immunosuppressant produced by Streptomyces tsukubaensis. Chemically, tacrolimus is designated as [3S-[3R*[E (1S*,3S*,4S*)], 4S*,5R*,8S*,9E,12R*,14R*,15S*,16R*,18S*,19S*,26aR*]]-5,6,8, 11,12, 13,14, 15, 16, 17, 18, 19,24,25,26,26a-hexadecahydro-5,19-dihydroxy-3-[2-(4-hydroxy-3-methoxycyclohexyl)-1-methylethenyl]-14,16-dimethoxy-4,10,12,18-tetramethyl-8-(2-propenyl)-15,19-epoxy-3H-pyrido[2,1-c][1,4] oxaazacyclotricosine-1,7,20,21 (4H,23H)-tetrone, monohydrate. Another exemplary calcineurin inhibitor is cyclosporine. Cyclosporine is a is a cyclic polypeptide immunosuppressant agent consisting of 11 amino acids. It is produced as a metabolite by the fungus species Beauveria nivea. Chemically, cyclosporine is designated as [R-[R*,R *-(E)]]-cyclic-(L-alanyl-D-alanyl-N-methyl-L-leucyl-N-methyl-L-leucyl-N-methyl-L-valyl-3-hydroxy-N,4-dimethyl-L-2-amino-6-octenoyl-L-α-amino-butyryl-N-methylglycyl-N-methyl-L-leucyl-L-valyl-N- methyl-L-leucyl).

In certain embodiments of any aspect disclosed herein, the immunosuppressant is an antiproliferative agent. Antiproliferative agents include, for example, mycophenolate mofetil and azathioprine. An exemplary antiproliferative is mycophenolate mofetil (MMF). MMF is the 2-morpholinoethyl ester of mycophenolic acid (MPA). The chemical name for MMF is 2-morpholinoethyl (E)-6-(1,3-dihydro-4-hydroxy-6-methoxy-7-methyl-3-oxo-5-isobenzofuranyl)-4-methyl-4-hexenoate. Another exemplary antiproliferative is azathioprine. Azathioprine is an imidazolyl derivative of 6-mercaptopurine. Azathioprine is chemically 6-[(1-methyl-4-nitro-1 H-imidazol-5-yl)thio]-1 H-purine.

In certain embodiments of any aspect disclosed herein, the immunosuppressant is a glucocorticoid. Glucocorticoids include, for example, prednisone, prednisolone, and methylprednisolone. An exemplary glucocorticoid is methylpredinsolone. The chemical name for methylprednisolone is pregna-1,4-diene-3,20-dione, 11,17,21-trihydroxy-6-methyl-, (6α, 11β)-and the molecular weight is 374.48.

In certain embodiments of any aspect disclosed herein, the immunosuppressant is an antibody. Antibodies include, for example, monoclonal antibodies such as basiliximab and OKT3 as well as polyclonal antibodies such as anti-thymocyte globulin. Basiliximab is a chimeric (murine/human) monoclonal antibody (IgGIκ), specifically binding to and blocking the interleukin-2 receptor α-chain (IL-2Rα, also known as CD25 antigen) on the surface of activated T-lymphocytes. OKT3, or muromonab-CD3, is a murine monoclonal antibody to the CD3 antigen of human T cells which functions as an immunosuppressant. Anti-thymocyte globulin comprises gamma globulin, primarily monomeric IgG, from hyperimmune serum of horses immunized with human thymus lymphocytes.

In certain embodiments of any aspect disclosed herein, the immunosuppressant is an mTOR inhibitor. mTOR inhibitors include, for example, rapamycin and everolimus.

It will be readily apparent to those skilled in the art that other suitable modifications and adaptations of the compositions and methods of the invention described herein may be made using suitable equivalents without departing from the scope of the invention or the embodiments disclosed herein.

The compounds, compositions, and methods described herein will be better understood by reference to the following examples, which are included as an illustration of and not a limitation upon the scope of the invention.

An immunosuppressive therapy may be provided as induction immunosuppressive therapy (i.e., short-term treatment approximately at the time of transplant) or as maintenance immunosuppressive therapy (i.e., post-transplant treatment). A goal of immunosuppressive therapy is to reduce the risk of allograft rejection.

An exemplary induction immunosuppressive therapy comprises an antibody. For example, one such induction immunosuppressive therapy comprises basiliximab or anti-thymocyte globulin.

An exemplary maintenance immunosuppressive therapy comprises a calcineurin inhibitor, a glucocorticoid, and an antiproliferative agent. For example, one such maintenance immunosuppressive therapy comprises a calcineurin inhibitor, methylpredinsolone, and MMF; another such maintenance immunosuppressive therapy comprises a calcineurin inhibitor, methylpredinsolone, and azathioprine.

### C. EXAMPLES

### Experimental Model and Subject Details

Clinical sample collection and analysis were approved by the Ethics Review Board of the Hôpital Erasme, Brussels, Belgium. Written informed consent was obtained from study participants. Clinical and demographic characteristics were collected from the patient's electronic medical records (Transkid-RedCap) (Table 1 and Table 2). Patients with detectable anti-HLA-A2 alloantibodies, whether or not these antibodies represented a DSA, were included in the study, a criterion based on development of assays for this specificity as a model and given its clinical prevalence. By necessity, this choice excluded recipients who were HLA-A2 antigen positive as well as individuals who were seropositive for other specificities but not HLA-A2, resulting in the exclusion of many patients diagnosed with DSA and experiencing AMR. The samples tested were those collected at the time that HLA-A2 seropositivity was first diagnosed. However, variability between subjects in the time between the induction of a response and its diagnosis is expected given screening required out-of-pocket costs to patients and was therefore optional. The median interval between transplant and DSA assessment yielding a positive result for HLA-A2 was 3,657 days (interquartile range (IQR) 2,246-7,244 days). Patients without detectable anti-A2 antibodies were included as controls. Anti-HLA-A2 antibodies were detected using clinical Luminex® single antigen bead assay according to the manufacturer's instructions (Immucor® Lifecodes). Following incubation with serum, Mean Fluorescence Intensity (MFI) of HLA antigen-coated beads was measured with a fluoroanalyser using xPonent software for data acquisition and Match It! Software (Immucor® Lifecodes) for data analysis. Positivity thresholds were defined according to the manufacturer's instructions. For individuals with renal transplant, AMR was diagnosed based on clinical parameters (serum creatinine and proteinuria) and renal transplant biopsy, when contributive. AMR histological characteristics were glomerulitis, peritubular capillaritis, microvascular inflammation and C4d immunostaining positivity. The median interval between diagnosis of AMR and HLA-A2-specific response detection (and test sample collection) was 1,483 days (IQR 1,286-2,228 days). Samples from patients with acute rejection were taken >1 yr after treatment with plasma exchange, IVIg, and corticoids. Patients with chronic rejection received no anti-rejection treatment.

TABLE 1
Cohort characteristics.

| | Anti-HLA-A2 | | | |
|---|---|---|---|---|
| | sensitized | | | |
| | group | | Control group | |
| Characteristics | (n = 32) | | (n = 18) | |
| Patient age - Years (P25-P75) | 45.5 | (28-57.5) | 48 | (41-61) |
| Patient gender - n (%) | | | | |
| Male | 12 | (37.5%) | 14 | (77.7%) |
| Female | 20 | (62.5) | 4 | (22.2%) |
| Sensitizing event -n (%) | | | | |
| Transplantation | 13 | (40.6%) | 5 | (27.8%) |
| Pregnancies | 6 | (18.8%) | 2 | (11.1%) |
| Blood derived product | 1 | (3.1%) | 1 | (5.6%) |
| transfusions | | | | |
| Left ventricular | 1 | (3.1%) | 0 | (0%) |
| assistance device | | | | |
| Unknown | 11 | (34.4%) | 2 | (11.1%) |
| No previous sensitizing event | 0 | (0%) | 8 | (44.4%) |
| cPRA % at the time of sample | 95.5 | (81.8-99) | 15.85 | (0-80.31) |
| collection (P25-P75) | | | | |
| Type of transplant among | | | | |
| transplanted patients -n (%) | | | | |
| KT | 25 | (78.1%) | 15 | (83.3%) |
| KPT | 1 | (3.1%) | 0 | (0%) |
| HT | 1 | (3.1%) | 0 | (0%) |
| PT | 1 | (3.1%) | 0 | (0%) |
| PLT | 0 | (0%) | 1 | (5.5%) |
| LKT | 0 | (0%) | 1 | (5.5%) |
| On transplant waiting list | 4 | (12.5%) | 1 | (5.5%) |
| Donor gender -n (%) | | | | |
| Male | 10 | (35.7%) | 13 | (76.5%) |
| Female | 16 | (57.1%) | 3 | (17.6%) |
| Unknown | 2 | (7.1%) | 1 | (5.9%) |
| Donor age among transplanted | 44 | (35-54) | 40.5 | (31.75-50) |
| patients (P25-P75)* | | | | |
| Donor category among | | | | |
| transplanted patients -n (%) | | | | |
| Deceased | 22 | (78.56%) | 15 | (88.2%) |
| Living | 3 | (10.7%) | 2 | (11.8%) |
| Not reported | 3 | (10.7%) | 0 | (0%) |
| Warm ischemia time | 32 | (27.5-38.5) | 36.5 | (24.5-43.25) |
| among transplanted | | | | |
| patients - minutes (P25-P75)* | | | | |
| Cold ischemia time | 19 | (13-21.5) | 20 | (6.95-20.5) |
| among transplanted | | | | |
| patients -hours (P25-P75)* | | | | |
| Recovery of graft function | | | | |
| among transplanted | | | | |
| patients - n (%) | | | | |
| Immediate | 20 | (71.4%) | 14 | (82.4%) |
| Delayed | 1 | (3.6%) | 2 | (11.8%) |
| Not reported | 7 | (25%) | 1 | (5.9%) |
| HLA A-B-DR mismatches | 3 | (2-3.5) | 3 | (2-4) |
| among transplanted | | | | |
| patients - n (P25-P75)* | | | | |
| Induction immunosuppressive | | | | |
| therapy among transplanted | | | | |
| patients -n (%) | | | | |
| No induction | 3 | (10.7%) | 2 | (11.8%) |
| Basiliximab | 6 | (21.4%) | 10 | (58.8%) |
| Thymoglobulin | 11 | (39.3%) | 3 | (17.6%) |
| OKT3 | 1 | (3.6%) | 1 | (5.9%) |
| Unknown | 7 | (25%) | 1 | (5.9%) |
| Maintenance | | | | |
| immunosuppressive therapy | | | | |
| among transplanted | | | | |
| patients - n (%) | | | | |
| CNI, MMF, mPDN | 19 | (67.8%) | 14 | (82.4%) |
| CNI, AZA, mPDN | 3 | (10.7%) | 0 | (0%) |
| Others | 0 | (0%) | 2 | (11.8%) |
| Not reported | 6 | (21.4%) | 1 | (5.9%) |
| AMR - n (%) | | | | |
| Unknown | 2 | (7.1%) | 2 | (11.8%) |
| No AMR | 13 | (46.4%) | 12 | (70.6%) |
| AMR | 13 | (46.4%) | 3 | (17.6%) |
| Acute AMR | 3 | (23.8%) | 2 | (66.7%) |
| Chronic AMR | 10 | (76.9%) | 1 | (33.3%) |

| | | | |
|---|---|---|---|
| Anti-HLA-A2 | | | |
| fucosylation - n (%) | | | |
| Low | 8 | (25%) | Glycans not |
| Medium | 8 | (25%) | determined |
| High | 8 | (25%) | |
| Undetermined | 8 | (25%) | |
| Time of anti-HLA-A2 | | | |
| antibodies detected among | | | |
| patients with AMR - n (%) | | | |
| Pre AMR | 2 | (15.4%) | |
| Post AMR | 11 | (84.6%) | |

*Data are given when available.

TABLE 2
DSA cohort characteristics.

| | Anti-HLA-A2 DSA | |
|---|---|---|
| Characteristics | sub-group (n = 13) | |
| Patient age - Years (P25-P75) | 30 | (21-44) |
| Patient gender - n (%) | | |
| Male | 9 | (64.3%) |
| Female | 12 | (35.7%) |
| Type of transplant - n (%) | | |
| KT | 12 | (92.3%) |
| KPT | 1 | (7.69%) |
| PLT | 0 | (0%) |
| LKT | 0 | (0%) |
| On transplant waiting list | 0 | (0%) |
| Donor gender -n (%) | | |
| Male | 6 | (46.1%) |
| Female | 7 | (53.9%) |
| Unknown | 0 | (0%) |
| Donor age - years (P25-P75) | 41 | (22-54) |
| Donor category - n (%) | | |
| Deceased | 11 | (84.6%) |
| Living | 1 | (7.7%) |
| Not reported | 1 | (7.7%) |
| Warm ischemia time - minutes (P25-P75)* | 32 | (28-33) |
| Cold ischemia time - hours (P25-P75)* | 20 | (16-26.5) |
| Recovery of graft function - n (%) | | |
| Immediate | 10 | (76.9%) |
| Delayed | 0 | (0%) |
| Not reported | 3 | (23.1%) |
| HLA A-B-DR mismatches - n (P25-P75)* | 2 | (2-3.5) |
| Induction immunosuppressive | | |
| therapy - n (%) | | |
| No induction | 1 | (7.7%) |
| Basiliximab | 2 | (15.4%) |
| Thymoglobulin | 5 | (38.5%) |
| OKT3 | 1 | (7.7%) |
| Unknown | 4 | (30.7%) |
| Maintenance immunosuppressive | | |
| therapy - n (%) | | |
| CNI, MMF, mPDN | 6 | (46.2%) |
| CNI, AZA, mPDN | 3 | (23.1%) |
| Others | 0 | (0%) |
| Not reported | 4 | (30.8%) |
| AMR - n (%) | | |
| Unknown | 0 | (0%) |
| No AMR | 6 | (46.2%) |
| AMR | 7 | (53.8%) |
| Acute AMR | 3 | (42.9%) |
| Chronic AMR | 4 | (57.1%) |
| Time of anti-HLA-A2 antibodies detected | | |
| among patients with AMR - n (%) | | |
| Post AMR | 7 | (100%) |
| Fucosylation profile among AMR | | |
| positive patients - n (%) | | |
| Low | 2 | (28.57%) |
| Medium | 4 | (57.14%) |
| High | 0 | (0%) |
| No glycan information | 1 | (14.28%) |
| Fucosylation profile among AMR | | |
| negative patients - n (%) | | |
| Low | 1 | (16.66%) |
| Medium | 0 | (0%) |
| High | 3 | (50%) |
| No glycan information | 2 | (33.33%) |

*Data are given when available.

Abbreviations used in Table 1 and Table 2: KT: Kidney transplant, KPT: combined kidney pancreas transplant, HT: heart transplant, PT: pulmonary transplant, PLT: combined pulmonary liver transplant, LKT: combined liver kidney transplant. CNI: calcineurin inhibitors; MMF: mofetil mycophenolate; mPDN methylprednisolone; AZA: Azathioprine; AMR: antibody mediated rejection.

### Method Details

Cloning and expression of recombinant HLA-A2 mAbs

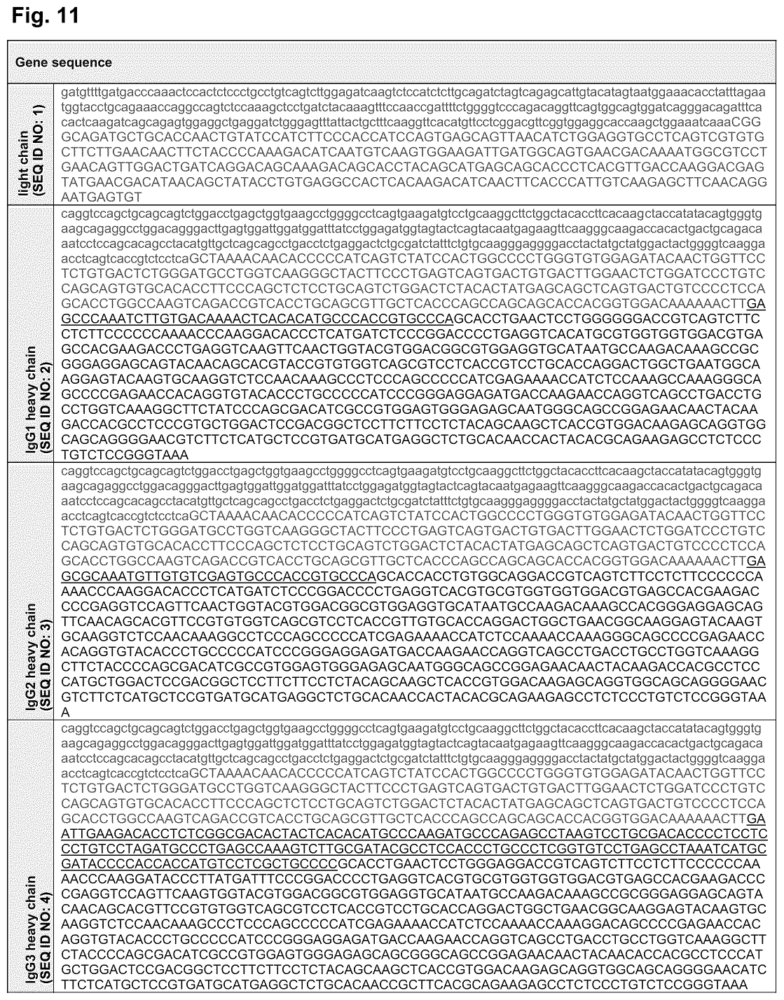

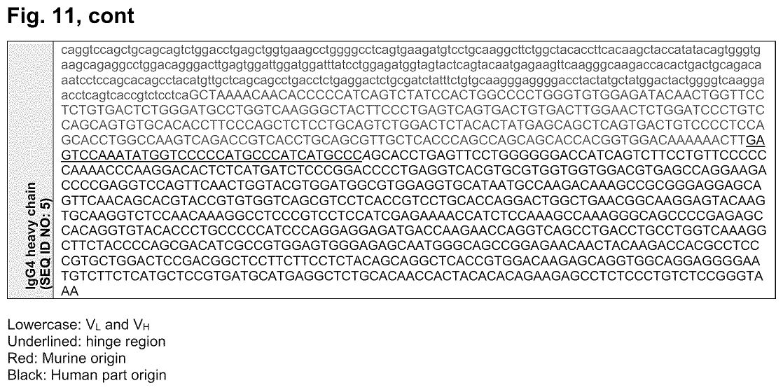

Variable domain gene sequences of the heavy and the light chain (VH and VL) of mouse HLA-A2 hybridoma cells (ATCC, HB-82 BB7.2 USA) were defined to support recombinant production of a panel of human subclass-switched chimeric antibodies. Briefly, mRNA was isolated from hybridoma cells using the RNeasy kit (Qiagen, Germany), and cDNA generated using the VRSO cDNA kit (ThermoFisher, USA). This cDNA was then amplified using degenerate primers to selectively amplify V_{H} and V_{L} regions, which were sequenced and cross-referenced and annotated using BLAST and IMGT-based tools. Verified V_{H} and V_{L} sequences were used to design gene blocks (Twist Biosciences, USA) that contained murine V_{H}, V_{L}, C_{L} and C_{H}1 domains paired with human hinge and C_{H}2 and C_{H}3 Fc domains for each IgG subtype (FIG. 11). These gene blocks were cloned by overlap extension into the pCMV expression plasmid.

Chimeric antibodies were transiently expressed via heavy and light chain plasmid co-transfection in HEK-expi293 cells, and purified using Protein A (IgG1, IgG2, and IgG4) or Protein G (IgG3) chromatography as previously reported. Afucosylated IgG1 and IgG3 were produced by adding 0.15 mM of 2-fluorofucose (2FF) substrate in the growth medium, as described.

### IgG Subclass and Response Magnitude Measurements

A custom multiplex assay was performed as previously described in order to define the total levels and subclass profiles of HLA-A2-, HLA-A1-(NIH Tetramer Facility, HLA-A*02:01 complexed with either CLGGLLTMV (SEQ ID NO: 6) peptide from Epstein Bar Virus membrane protein or GLCTLVAML (SEQ ID NO: 7) peptide from Epstein Bar Virus mRNA export factor ICP27, HLA-A*01:01 complexed with VTEHDTLLY (SEQ ID NO: 8) from cytomegalovirus pp50), and HSV-gD-(Immune Technology, USA) specific antibodies. Briefly, antigen-coupled microspheres were diluted in Assay Buffer (PBS+0.1% BSA+0.05% Tween20), and mAb or serum, followed by washing and detection with R-phycoerythrin (PE)-conjugated anti-Human IgG (Southern Biotech, USA), anti-mouse IgG (Biolegend, USA) or anti-human IgG1 (Southern Biotech, USA), IgG2 (Southern Biotech, USA), IgG3 (Southern Biotech, USA) and IgG4 (Southern Biotech, USA), respectively. These detection reagents have been characterized recently for specificity and sensitivity across IgG allotypes. Median fluorescent intensities (MFI) were acquired on a FlexMap 3D (Luminex, USA).

### Affinity Purification of HLA-A2 Specific Antibodies

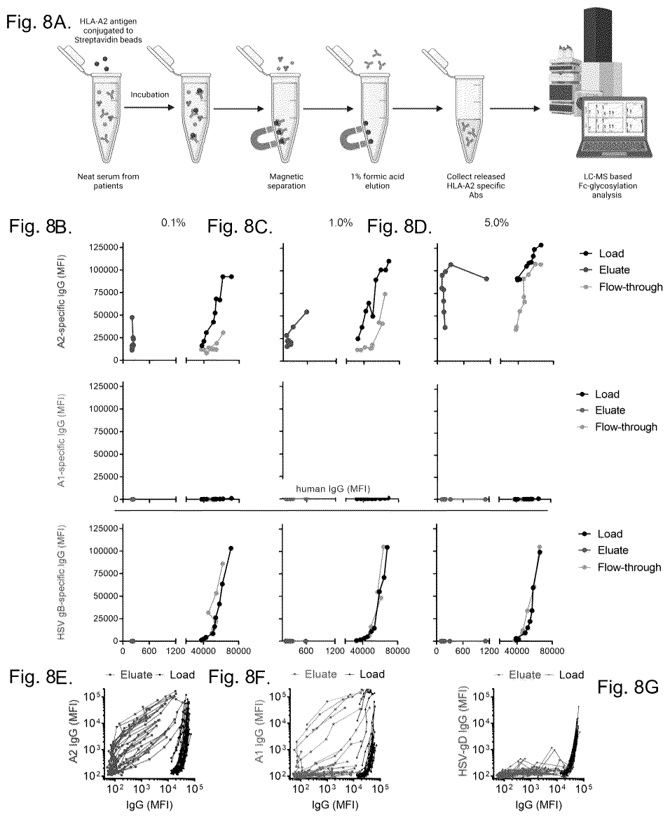

HLA-A2-specific antibodies were purified from human sera using magnetic, antigen-conjugated beads (FIGS. 8A-8G; Table 3). Magnetic streptavidin coated beads (NEB, S1420S) were incubated with biotinylated HLA-A2. Briefly, 50 μL streptavidin beads were washed with wash buffer (0.5 M NaCl, 20 mM Tris-HCl (pH 7.5), 1 mM EDTA) followed by incubation with 20 μg biotinylated HLA-A2 for 2 hours at room temperature or overnight at 4° C. After washing five times, beads were blocked using 200 μL heat inactivated FBS for 2 hours. For purification, 150 μL of beads were co-incubated with 50 μL of sera for 3 hours on a rotational mixer, followed by three washes using PBS-TBN (PBS-1X, 0.1% BSA, 0.02% Tween 20, 0.05% sodium azide, pH 7.4). HLA-A2-specific antibodies were eluted by resuspending beads in 50 μL of 1% formic acid (pH 2.9) and incubating on a rotational mixer for 10 min at room temperature. Supernatant was withdrawn and 20 μL of 0.5 M sodium phosphate dibasic was added to each tube to neutralize the pH. The resulting eluate was split into two parts and used for LC-MS-based IgG Fc glycosylation analysis as well as for enrichment confirmation analysis, as described above.

TABLE 3
Details of HLA molecules used for the
purification of HLA-specific IgGs

| Specificity | Allele | Loaded peptide | Origin |
|---|---|---|---|
| HLA-A1 | HLA | VTEHDTLLY | CMV, pp50 |
| | A*01: 01 | (SEQ ID NO: 8) | Influenza A |
| | | CTELKLSDY | virus, Nucleo- |
| | | (SEQ ID NO: 9) | protein, 44-52 |
| HLA-A2 | HLA | CLGGLLTMV | EBV membrane |
| | A*02: 01 | (SEQ ID NO: 6) | protein, |
| | | GLCTLVAML | 42-434 |
| | | (SEQ ID NO: 7) | EBV, mRNA |
| | | | export factor |
| | | | ICP27, 300-308 |

### Affinity Purification of Total IgG Antibodies

Total IgG was captured from plasma/serum using Protein G Sepharose Fast Flow 4 beads (GE Healthcare, Uppsala, Sweden) according to established procedures. Briefly, affinity beads were span down, stripped from their supernatants and resuspended in 1× phosphate-buffered saline (PBS) in a 1:24 bead slurry-to-PBS ratio. Next, 50 μL suspended beads were pipetted into each well of a 96-well filter plate (Orochem Technologies, Naperville, IL). Following three washing steps with 1× PBS, 1 μL of each plasma sample premixed in 20 μL 1× PBS were applied to the wells. Then, the plate was sealed and incubated at room temperature on a horizontal shaker at 1000 rpm for an hour, after which samples were subjected to consecutive washing steps by 1× PBS and water (thrice each). Consecutively, 100 μL of 100 mM formic acid (Sigma-Aldrich, Steinheim, Germany) solution was added into each well and the plate was incubated for 5 minutes on a horizontal shaker at 1000 rpm, which was followed by centrifuge-aided elution (100 g for 1 min) into a 96-well collection plate. Subsequently, purified total IgGs were dried by vacuum centrifugation and subjected to overnight tryptic digestion at 37° C. following their resuspension in 20 μL 50 mM ammonium bicarbonate and 20 μL sequencing grade trypsin solution (25 ng per sample: Promega Corporation, WI, Madison). Following tryptic digestion, the purified total IgG glycopeptides were stored at −20° C. until LC-MS analysis.

### Preparation of HLA-A2-Specific Antibodies for LC-MS-Based Fc Glycosylation Analysis

Affinity purified HLA-A2-specific IgG dried by vacuum centrifugation and subsequently redissolved in 20 water and 7 μL 1× PBS, which resulted in a pH of 8. Next, samples were subjected to tryptic digestion overnight at 37° C. using sequencing grade trypsin (25 ng per sample). Then, tryptic HLA-A2 specific IgG glycopeptides were enriched and desalted by reversed-phase solid phase extraction (Chromabond C18ec beads (Marcherey-Nagel, Düren, Germany), similarly to a previous report. A 20 mg/mL suspension was obtained in 50% acetonitrile (ACN; Biosolve, Valkenswaard, Netherlands), of which 250 μL was added to the wells of a 96-well filter plate (Orochem Technologies Inc., Naperville, IL). The beads were activated by consecutive washing steps with 80% ACN 0.1% trifluoroacetic acid (TFA; Merck, Darmstadt, Netherlands), 50% ACN 0.1% TFA and finally three times 0.1% TFA (200 μL each). Then, samples were added to the filterplate in 0.1% TFA and shaken for 10 min at 500 rpm on a horizontal shaker, followed by three washing steps by 0.1% TFA (100 μL each). The bound Fc glycopeptides were firstly eluted with 100 μL of 18% ACN 0.1% TFA, and secondly with 100 uL of 50% ACN 0.1% TFA into separate 96-well V-bottom plates. Each elution step was preceded by 5 minutes incubation at 500 rpm on a horizontal shaker in the respective eluent. Subsequently, the desalted, matching HLA-A2-specific IgG glycopeptide samples from the two elution plates were pooled, and then dried by vacuum centrifugation. The dried samples were resuspended in 40 μL of 25 mM ammonium bicarbonate and stored at −20° C. until LC-MS analysis.

### LC-MS Based Fc Glycosylation Analysis

Glycopeptides were separated with an Ultimate 3000 RSLCnano high-performance liquid chromatography system (Thermo Scientific, Waltham, MA) equipped with an Acclaim PepMap 100 trap column (100 μm×20 mm, 5 um particle size; Thermo Scientific) and an Acclaim PepMap RSLC C18 nano-column (75 μm×150 mm, 2 um particle size) analytical column. Five hundred nL of total IgG and two hundred nL of HLA-A2-specific IgG was injected and separated with a gradient from 97% solvent A (0.1% formic acid in water) and 3% solvent B (95% ACN) down to 27% solvent B, at a flowrate of 700 nL/min over 15 minutes. The LC-MS system was hyphenated to a maXis HD quadrupole time-of-flight mass spectrometer (Bruker Daltonics, Billerica, MA) via an electrospray ionization interface, which was equipped with a CaptiveSpray nanoBooster using ACN-enriched nitrogen gas (at 0.2 bar pressure and a dry gas flow rate of 3 L/min). A frequency of 1 Hz was used for recording the spectra in the m/z range of 550-1800 in positive ion polarity mode. The transfer time was set to 130 us, the pre-pulse storage time to 10 us, while the collision energy was set to 5 eV. This method allowed unambiguous identification of IgG Fc glycopeptides in a subclass specific manner based on accurate mass (MS1) and specific migration positions in liquid chromatography.

### LC-MS Data Processing

Data processing was performed according to established procedures. Briefly, mzXML files were generated from raw LC-MS spectra using MSConvertGUI (ProteoWizard, Palo Alto, CA) and an in-house developed software, LaCyTools was used for chromatogram alignment, calibration, and targeted data extraction. The targeted glycopeptide extraction list included the 2+and 3+charge states and was generated by manual annotation of the mass spectra and based on literature. A commercially available polyclonal human IgG standard (isolated from normal human plasma; Athens Research & Technology, Athens, GA) and due to limited sample amount, plasma of a single HLA-A2 positive individual were prepared and measured in triplicates to assess robustness of the HLA-A2-specific method, resulting in an average intra-plate coefficient of variation (CV) of 3.7%. Overall method robustness for the total IgG method was assessed by preparing and measuring triplicate plasma samples of six HLA-A2 positive individuals, resulting in an average intra-plate CV of 1.4%. Plasma from seven HLA-A2 negative individuals were used as negative controls. All spectra below the average intensity plus once the standard deviation of negative controls was excluded from further analysis. Inclusion of an analyte for final data analysis was based on quality criteria including signal-to-noise (>9), isotopic pattern quality (<25% deviation from the theoretical isotopic pattern), and mass error (within a+20 ppm range). Furthermore, only analytes present in at least 1 out of 4 HLA-A2-specific IgG spectra (25%) were included for relative quantification.

### FcγR Binding Assay

The FcγR binding profiles of polyclonal HLA-A2-specific antibodies were defined using the Fc Array assay described previously. Briefly, HLA-A2 coated microspheres were generated as described above for the multiplex assay, serum antibodies were detected with FcγRIIIa tetramers formed by mixing biotinylated FcγRIIIa V158 with a 1/4^{th} molar ratio of Streptavidin-RPE (Agilent Technologies, USA).

### FcγRIIIa Signaling Assay

As a surrogate for the ADCC potential of the serum-derived and recombinant HLA-A2 specific antibodies, FcγRIIIa signaling was measured by using a Jurkat Lucia NFAT reporter cell line (InvivoGen, USA) in which cross-linking of antigen-bound antibodies and FcγRIIIa leads to the secretion of luciferase into the cell culture supernatant. Levels of Lucia luciferase secreted can then be directly measured by bioluminescence. Briefly, a clear flat bottom, high binding 96 well plate (Corning, USA) was coated with either 1 μg/mL NeutrAvidin (ThermoFisher Scientific, USA) or 1 μg/mL biotinylated HLA-A2 antigen via incubation at 4° C. overnight. The plates were then washed with 1X phosphate-buffered Saline (PBS) plus 0.05% Tween 20 and blocked with 1× PBS plus 2.5% bovine serum albumin (BSA) for 1 hour at room temperature. HLA-A2 antigen coated plates were directly used, whereas the plates coated first with NeutrAvidin were washed and incubated with 2 μg/mL biotinylated HLA-A2 antigen for 1 hour at room temperature. After washing, 200 μL of serum diluted 1:100 and 100,000 cells/well in growth medium lacking antibiotics were added and incubated at 37° C. for 24 hours. Alternatively, for HLA-A2 mAbs, the plates were first incubated with mAb for 3 hours at room temperature, washed and then was incubated with cells. The following day, 25 μL of supernatant was drawn from each well and then transferred to an opaque, white, clear bottom 96-well plate (Thomas Scientific, USA) to which 75 μL of freshly prepared QuantiLuc (InvivoGen, USA) was added. Luminescent was measured immediately on a SpectraMax Paradigm plate reader (Molecular Devices, USA) using 1 s of integration time for collecting light. The reported values are the means of three kinetic readings collected at 0, 2.5 and 5 minutes. Treatment of reporter cells with cell stimulation cocktail (ThermoFisher Scientific, USA) was used as a positive control, and an a4B7-specific mAb was used as a negative control. Baseline control wells contained assay medium instead of antibody sample.

### Antibody Dependent Cellular Cytotoxicity (ADCC) Assay

ADCC activity of the HLA-A2 mAbs were assessed by a plate-based assay using an adaptation of a method described previously. Briefly, A375 cells which expressed GFP, obtained by transducing A375 cells (ATCCR, USA) with Ppy RE9-GFP retroviral backbone (50,000 cells/well) were added to the titrated HLA-A2 mAbs in a total of 100 μL of complete assay medium (RPMI-1640 media (Corning, USA) supplemented with 10% heat-inactivated FBS (Biowest), 1 mM sodium pyruvate (Corning, USA), 1X MEM nonessential amino acids (Corning, USA) and 1X Penicillin Streptomycin (Corning, USA)), in a sterile, U-bottom 96 well suspension culture plate (Greiner Bio-one). The plate was then incubated at 37° C. at 5% CO₂ for 30 min. NK-92 human NK cells (NantKwest, formerly Conkwest) were used as effector cells for the assay. 50 μL of NK-92 cells were then added at an effector to target ratio (E:T) of 2:1. Cells were co-cultured at 37° C. at 5% CO2 for 3 hours. After 3 hours, contents of the wells were transferred to a 96 well V-bottom plate (USA Scientific, USA). The cells were washed with ice-cold 1X PBS following centrifugation at 400 g for 5 min at 4° C. After that, the cells were stained with live/dead fixable violet (Invitrogen, USA) for 30 minutes in ice according to the manufacturer's instructions, and then were fixed with 4% Paraformaldehyde (Electron Microscopy Sciences, 15710) at 4° C. in dark for 30 minutes, washed with PBSF (1X PBS supplemented with 0.1% BSA), and resuspended in 100 μL of PBSF. Data were acquired on a Novocyte flow cytometer and analyzed using Flowjo V10. The FSC-H and SSC-H parameters were used to gate the cells. The target population was gated as the GFP⁺ population based on SSC-H vs FITC-H biplot. The proportion of cell death was determined based on the percentage of GFP+/Violet+ cells. Data were analyzed in GraphPad Prism.

### FcγR Affinity Measurements

High Precision Streptavidin 2.0 (SAX2) biosensor tips (Sartorius) were used to determine the kinetics of HLA-A2-specific IgG binding to human FcγRIIIa and FcγRI by biolayer interferometry (BLI) on an Octet (Forte Bio) instrument. Streptavidin (SA) biosensor tips were first loaded with HLA-A2 antigen, followed by HLA-A2 antibody, and finally dipped into FcγR to study the antibody-receptor kinetics. Biosensors were equilibrated with 0.05% Tween-PBS for 60 s, loaded with 0.25 μg/mL biotinylated HLA-A2 antigen (in 0.05% Tween-PBS) until they reached the threshold of 1 nm, and then dipped into 0.05% Tween-PBS to reach baseline for 60 s. After that, the biosensors were loaded with 3 μg/mL of recombinant HLA-A2 IgG (in 0.05% Tween-PBS) for 600 s, dipped into 0.05% Tween-PBS to reach baseline for 60 s, and then dipped into 5 uM monomeric FcγRIIIa for 180 s or 292 nM FcγRI for 300 s for association. To improve signal to noise, serum IgG was purified using Melon gel (Thermo) according to the manufacturer's instructions. Polyclonal serum IgG was loaded for 2,400 s. For FcγR dissociation rate (k_{d}) determination, two SAX2 biosensor tips were used, one for the measurement and one as a reference to subtract background signal changes due to dissociation of other components, such as dissociation of HLA-A2-specific IgG from biotinylated HLA. Reference biosensors loaded with IgGs were dipped into buffer (0.05% Tween-PBS) instead of receptors. Finally, the sensors were dipped into 0.05% Tween-PBS for 180 s and 300 s for the dissociation of FcγRIIIa and FcγRI, respectively. Data analysis.

### Quantification and Statistical Analysis

Statistical analysis was performed in GraphPad Prism version 9. Replicates and statistical tests used are reported in figure legends. A nonlinear regression using a one phase exponential decay model was used to fit the dissociation curve and calculate off-rate.

### Results

HLA-specific antibody responses were investigated in a cohort consisting of individuals with reported clinical HLA-A2 reactivity (n=32) as detected with a single bead antigen assay and controls (n=18) with no HLA-A2 reactivity. Among anti-A2-sensitized patients, 28 had received an organ transplant, including 26 kidney transplants and 2 other organs, and 4 patients were on a transplant waiting list (Table 1). A2 sensitization was related to previous or current transplantation in 13 patients, pregnancy in six, blood transfusion in one, and left ventricular assistance device in one, whereas the sensitizing event was unknown in 4 patients. Thirteen out of the 28 (46%) patients with a transplant (Table 1) and 7 out of the 13 (54%) patients who were DSA positive (A2 sensitization in a patient transplanted with an HLA-A2 graft) (Table 2) were diagnosed with either acute or chronic AMR. Among controls, nine patients had no detectable anti-HLA antibodies, and nine had detectable antibodies against non-HLA-A2 antigens. Seventeen controls had received an organ transplant, and one was on a transplant waiting list.

### IgG Subclass Distribution of HLA-Specific Antibodies

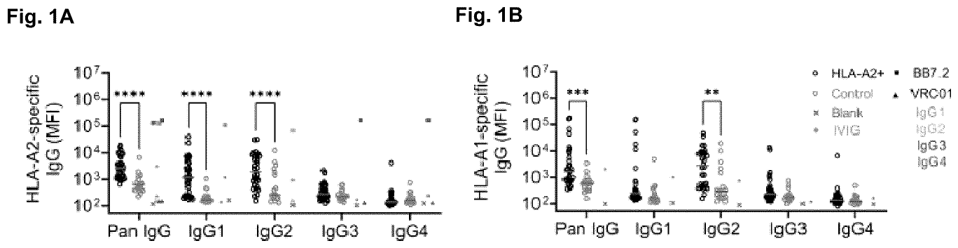

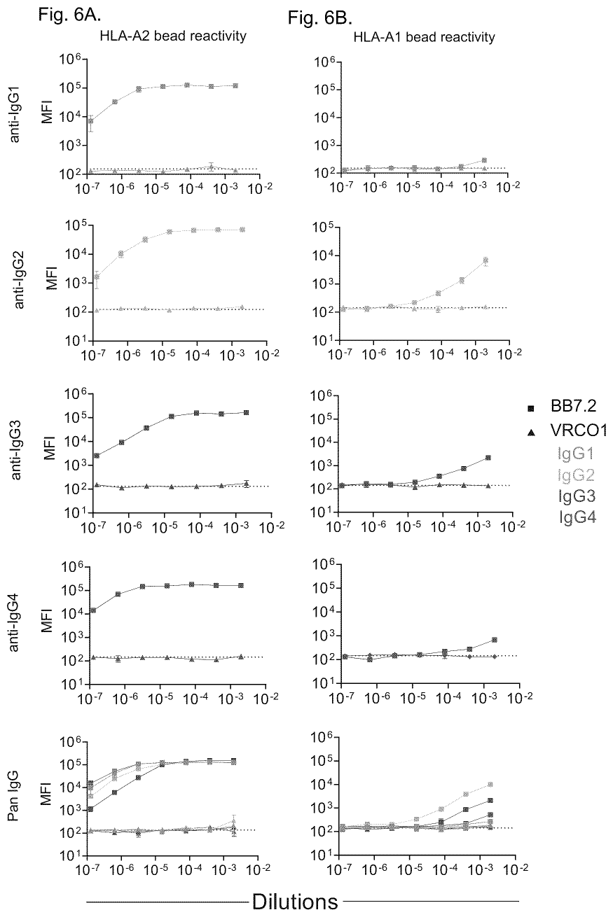

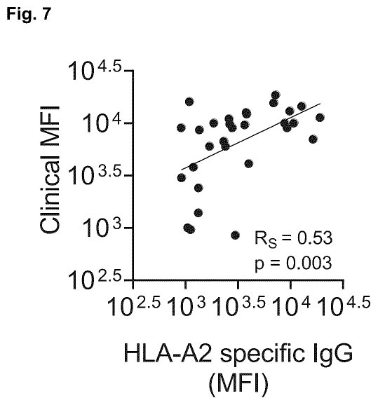

We first sought to characterize the subclasses of HLA-specific IgGs in HLA-A2-sensitized and control subjects, using research-grade single-antigen bead immunoassays. Briefly, single antigens (intact HLA-A*02:01 or HLA-A*01:01 monomers) were captured on microspheres, and antigen binding of sera samples was measured by multiplex assay. In contrast to the HIV-specific antibody VRC01 (negative control), the HLA-A2-specific monoclonal antibody BB7.2 (positive control), which was evaluated in each human IgG subclass, showed specific binding to HLA-A2 (FIG. 1A) and appropriate staining by IgG subclass detection reagents (FIG. 6A). In contrast to the negative control monoclonal antibody (mAb), both pooled human serum Ig (IVIG) and serum from individual control subjects often showed signal considerably above background. While this profile may be consistent with the widespread prevalence of anti-HLA antibodies among healthy individuals, and responses among subjects clinically defined as HLA-A2 seropositive were significantly elevated compared with controls, there was a considerable overlap in distributions. Nonetheless, there was a distinct difference observed in the subclass distribution between the two groups, with the HLA-A2-seropositive group generally exhibiting a wide range of total HLA-A2 antibodies compared with the control samples, which tended to show lower levels (FIG. 1A). The wide distribution of the HLA-A2-specific IgG signal was consistent with reported values from clinical testing (FIG. 7), with some samples exhibiting moderate to very low HLA-A2 reactivity. HLA-A2-reactive antibodies predominantly belonged to the IgG1 and IgG2 subclasses, but a few subjects also exhibited elevated levels of IgG3 and IgG4; these individuals typically had high levels of total HLA-A2-specific IgG.

A similar analysis of HLA-A1-specific antibody levels was performed. The HLA-A2-seropositive group also showed elevated levels of HLA-A1 antibodies compared with the control group (FIG. 1B), again, consistent with clinical single-antigen bead assay results. Interestingly, HLA-A1-specific antibodies predominantly belonged to the IgG2 subclass, with some samples exhibiting higher levels of IgG1, IgG3, and IgG4 subclasses.

### Affinity Enrichment Yields HLA-A2-Specific Antibodies

A microscale purification method was successfully developed and employed for the purification of a control murine HLA-A2-specific mAb spiked into IVIG (FIGS. 8A-8G). Purified antibodies showed elevated reactivity to HLA-A2 antigen, but not to HLA-A1 or to a herpes simplex virus (HSV) antigen, which were used as negative controls, confirming the purity of the antibodies. Having vetted the purification process, HLA-A2-specific antibodies were then purified from serum of HLA-A2-antibody-seropositive patients. The purified antibody fractions showed elevated reactivity to HLA-A2 but not to HSV. Despite generally being thought not to possess shared epitopes, the HLA-A2-enriched fraction showed some cross-reactivity with HLA-A1 for a few subjects who were generally strongly positive for both HLA-A1 and HLA-A2 antibodies (based on clinical single-antigen bead assay) (FIGS. 8A-8G).

### HLA-A2-Specific Antibodies Exhibit Variable Degree of Afucosylation

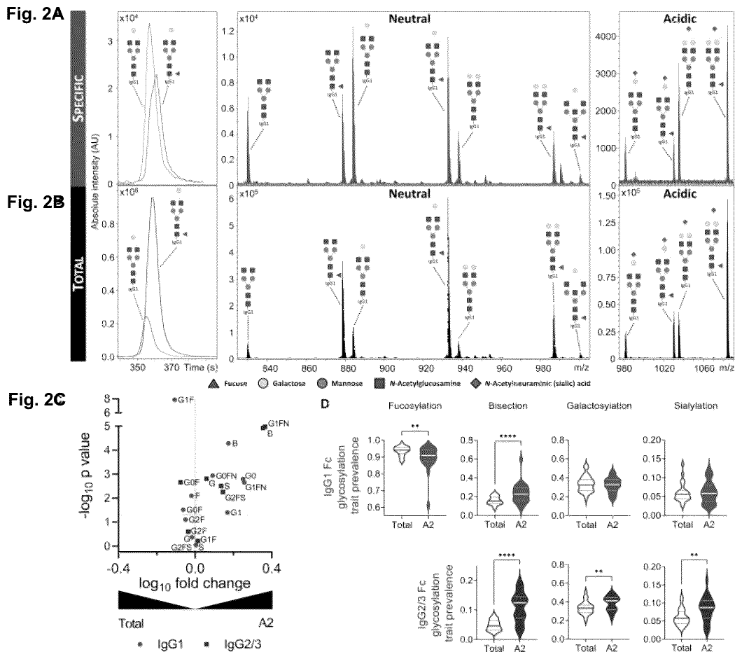

Having confirmed the largely selective enrichment of HLA-A2-reactive antibodies, next we analyzed Fc glycosylation of both HLA-A2-specific and total serum IgG among HLA-A2-seropositive individuals. Fc glycosylation analysis revealed differences both on the level of individual glycoforms (FIGS. 2A-2C) and overall glycosylation traits including levels of fucosylation, bisection, galactosylation, and sialyation (FIG. 2D) between HLA-A2-specific and total serum IgG1. Of note, while HLA-A2-specific antibody Fc fucosylation of some individuals was comparable to that of total IgG1, others exhibited fucosylation levels reduced by 5%-15%, or even 30%, compared with their total IgG1 counterpart (FIG. 2D). HLA-A2-specific IgG2/3 was characterized by higher bisection, galactosylation, and sialylation than total serum IgG2/3, but no afucosylated IgG2/3 glycopeptides were detected (FIG. 2D). Consistent with multiplex assay data, antigen-specific IgG4 responses were generally too low to be robustly characterized by liquid chromatography with mass spectrometry (LC-MS). While IgG2 and IgG3 glycopeptides could not be distinguished by MS due to their identical molecular mass, the high levels of antigen-specific IgG2 and low levels of IgG3 observed suggest that the antigen-specific IgG2/3 glycosylation signatures were dominated by IgG2.

Afucosylated HLA-A2-Specific mAbs Exhibit Enhanced FcγRIIIa Signaling and ADCC Activity

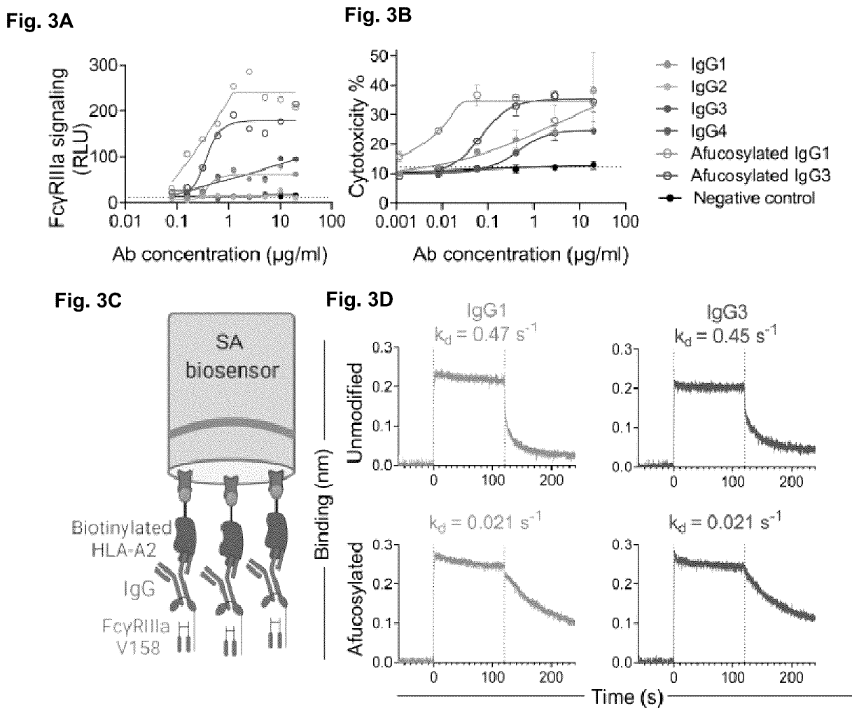

To study the FcγR signaling characteristics of HLA-A2-specific antibodies, we produced afucosylated BB7.2 IgG1 and IgG3 mAbs and assessed signaling driven by ligation of FcγRIII, a receptor typically expressed by NK cells, in a reporter cell line as a surrogate for ADCC activity. IgG1 and IgG3 isotypes of BB7.2 were found to show FcγRIIIa ligation and signaling activity, while neither the control IgG1 subclass nor the IgG2 and IgG4 subclasses of BB7.2 showed activity above baseline (FIG. 3A). As expected, afucosylated IgG1 and IgG3demonstrated considerably enhanced FcγRIII signaling activity compared with their non-glycoengineered counterparts.

Lastly, we evaluated the cytotoxic activity of HLA-A2-specific antibodies by measuring the antibody-dependent killing of HLA-A2+A375 cells, a human melanoma cell line, mediated by NK-92, a human NK cell line. Similar to the FcγRIIIa signaling activity, afucosylated IgG1 and IgG3 demonstrated higher ADCC activity compared with their non-glycoengineered counterparts and a negative control antibody across a range of antibody concentrations (FIG. 3B).

Enhanced FcγRIII Signaling and ADCC Activity are Associated with Slow Antibody Dissociation

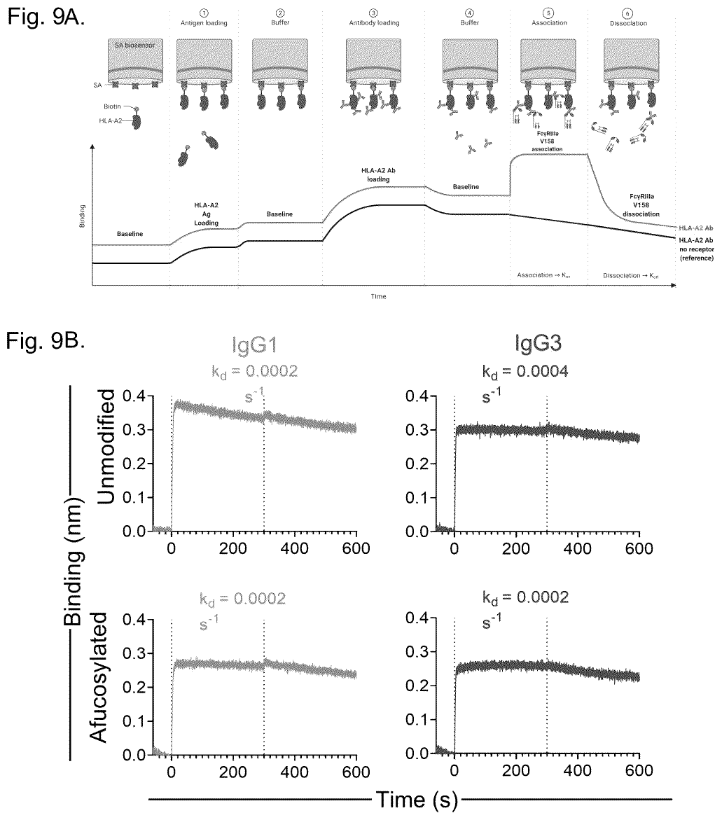

To address the mechanism of improved FcγRIIIa signaling and ADCC activity, we next sought to characterize the interaction between HLA-A2-specific antibodies and FcγRIIIa using biolayer interferometry (BLI) (FIG. 3C and FIGS. 9A-9B), a label-free technique commonly used to study the affinity and kinetics of protein-protein interactions. Antibodies bound to HLA-A2 antigen were allowed to bind (t=0-120 s) and then dissociate (t=120-240 s) from FcγRIIIa V158. All mAbs exhibited the fast on rate typical of FcγR and showed similar levels of FcγRIIIa-binding signal (FIG. 3D). In contrast, dissociation rates (K_{D}) varied by more than an order of magnitude between fucosylated and afucosylated mAbs (FIG. 3D). We found that afucosylated IgG1 and IgG3 had slower dissociation rates (mean K_{D}=0.021 s⁻¹) compared with unmodified forms (mean K_{D}=0.46 s⁻¹). This difference in dissociation rate was specific for FcγRIIIa as fucosylation did not influence FcγRI binding (FIGS. 9A-9B), as expected.

Serum HLA-A2-Specific Antibody Fucosylation Associates with FcγRIIIa Dissociation and Ligation

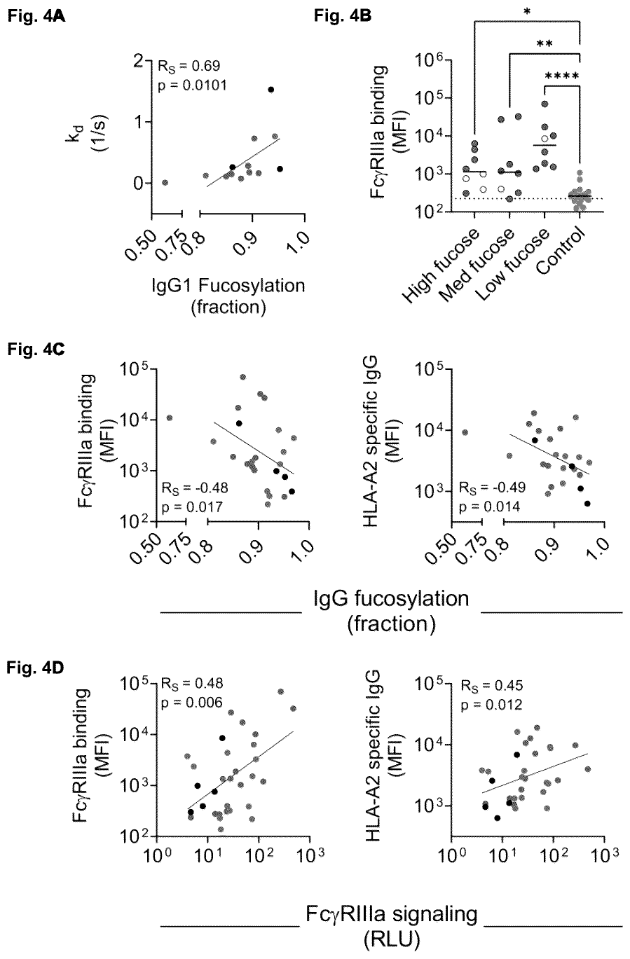

Since afucosylated HLA-A2-specific mAbs had slower dissociation from FcγRIIIa compared with their unmodified counterparts, we next studied the dissociation profiles of polyclonal HLA-A2-specific antibodies in human serum and found the FcγRIIIa dissociation rate to be positively associated with fucosylation (FIG. 4A). The variable off rates observed among differentially fucosylated serum-derived HLA-A2-specific antibodies suggested that FcγRIIIa ligation and signaling profiles of these HLA-A2-specific antibodies might also vary. To test this possibility, HLA-A2-seropositive subjects were split into tertiles (high, medium, and low) based on their HLA-A2-specific antibody fucosylation levels (FIG. 4B). A multiplex assay was conducted in which HLA-A2-specific antibodies were evaluated for their ability to bind FcγRIIIa tetramers.

Although there was no significant difference in FcγRIIIa binding between high, medium, and low fucose tertiles, HLA-A2-specific antibodies in each of these three categories had higher FcγRIIIa binding compared with negative controls, as exemplified by the observed negative correlations (FIG. 4B). In line with observations on variably fucosylated mAbs, these results demonstrate that human serum-derived HLA-A2-specific antibodies with low fucosylation exhibit improved FcγRIIIa binding.

Serum HLA-A2-Specific Antibody Fucosylation Associates with Functional Activity

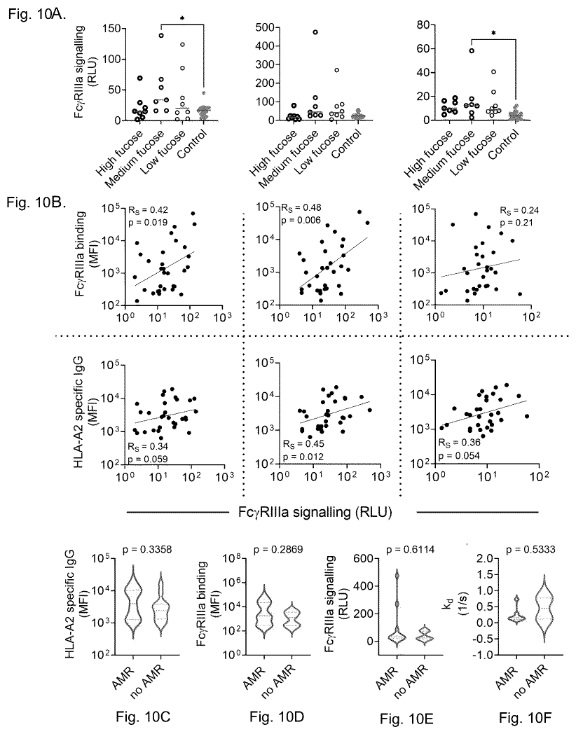

To understand how antibody fucosylation level was related to FcγRIIIa signaling, a limited correlation analysis was performed. Both HLA-A2-specific antibody response magnitude and the FcγRIIIa ligation activity determined by multiplex assay were negatively associated with fucosylation (FIG. 4C) and positively associated with FcγRIIIa signaling (FIG. 4D). Despite the low signaling activity observed across experimental replicates in the reporter cell line assay (FIG. 10A), measured relationships between FcγRIIIa signaling were consistently correlated with polyclonal HLA-A2-specific antibody response magnitude and FcγRIIIa binding (FIG. 10B).

HLA-A2-Specific Antibody Afucosylation was Correlated with Clinical AMR

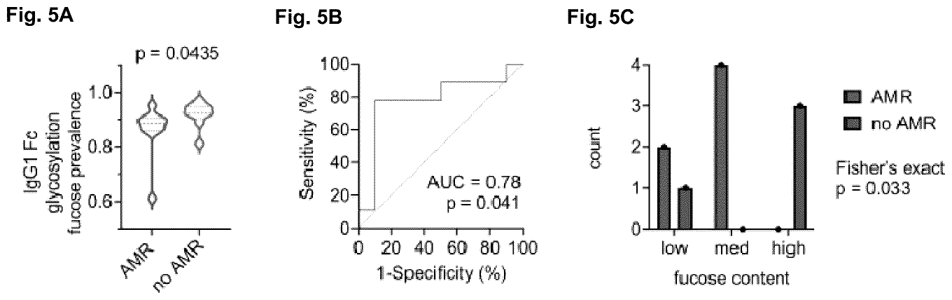

To explore how HLA-A2-specific antibody characteristics relate to transplantation outcomes, patient data were analyzed based on AMR status. Subjects with AMR tended to have high HLA-A2 antibody responses and low HLA-A2-specific IgG1 fucosylation, thereby showing high FcγRIIIa binding, high signaling activity, and slower dissociation from FcγRIIIa (FIG. 4). As a more direct test, we analyzed HLA-A2-specific antibody features between subjects with and without AMR. Whether or not anti-HLA-A2 represented a DSA, individuals with AMR showed significantly lower levels of HLA-A2-specific IgG1 fucosylation compared with the individuals who did not have clinically defined AMR (FIG. 5A). Receiver operating characteristic (ROC) curve analysis performed in the anti-A2-sensitized cohort (FIG. 5B) to define the accuracy of AMR status classification based on IgG1 fucose prevalence achieved an area under the curve (AUC) of 0.78, indicating a good discrimination for individuals with different AMR outcomes, suggesting that a low level of HLA-A2-specific antibody fucosylation may be a marker of AMR in patients who have had a kidney transplant. Other features, including HLA-A2-specific antibody response magnitude, did not show such differences according to AMR status (FIGS. 10C-10F).

In order to further examine the correlation between afucosylation of DSAs and clinical AMR outcomes, we analyzed subjects who had anti-HLA-A2 as a DSA. Patients who developed AMR had “low” and “moderate” fucose content of HLA-A2-specific DSAs, whereas patients who did not develop AMR had “high” fucose content of HLA-A2-specific DSAs (FIG. 5C). Collectively, these data suggest that the phenotypic variability among anti-HLA-A2 antibody responses may have an important influence on organ transplant outcomes.

### Discussion

The last two decades have witnessed a considerable increase in understanding immunological processes related to organ rejection, which has contributed to reduction in the incidence of acute rejection and improvement in short-term graft survival. Despite the advances in immunosuppression strategies, HLA matching, and better management of acute rejection, the prognosis for long-term graft outcome remains one of the biggest challenges in the field of organ transplantation. Given the well-established importance of qualitative aspects of the antibody response in other settings, consideration of antibody subclass, Fc glycosylation, and FcγR binding in the context of renal transplant outcomes will define the importance of these features in mediating functions that escalate the risk of organ rejection. Discovery of robust relationships between features of DSAs and transplant outcomes has the potential to drive revision of clinical strategies of organ allocation as well as development of novel interventions.

To date, relatively few studies have looked at DSA subclass and Fc glycosylation in kidney transplant recipients. Challenges in DSA purification from plasma/serum appear to have posed a major hurdle to its glycosylation analysis, resulting in a focus on characterization of total serum IgG glycosylation. In this study, we employed a microscale purification method to purify HLA-A2-specific antibodies from patient sera and set out to evaluate IgG subclass and Fc glycosylation profiles thereof. Both of these features regulate Fc-FcγR interactions, which in turn potentially relate to pathogenicity and graft rejection.

Beyond the complexity of the many processes associated with allograft injury and rejection, phenotypic changes in DSA over time have also been observed, complicating the understanding of the pathophysiology of rejection. What has become clear is that DSA titers, subclass profiles, or Clq binding activity alone are insufficient to accurately predict the course of AMR, and the field remains in need of reliable prognostic biomarkers to guide treatment, optimally allocate organs, and reduce the risk of rejection in DSA-sensitized recipients.

Though not strongly associated with outcomes of organ rejection, IgG subclass is known to strongly modify the ability of antibodies to drive complement deposition and recruit the innate immune effector cells that mediate clearance of opsonized particles. HLA-A2-specific responses in clinically seropositive subjects were predominantly IgG1 and IgG2. High levels of IgG1 antibodies suggest the possibility of elevated Fc-mediated effector functions, such as phagocytosis, ADCC, and complement deposition, which might lead to severe graft injury and transplant rejection. The prevalence of HLA-A1-specific IgG2 antibodies in this population suggests that the IgG subclass profile can vary from one HLA antigen to another even within a given individual, highlighting the potential importance of integrating profiles across diverse specificities and characterizing features of anti-HLA antibodies beyond titer.

Antibody effector functions are highly affected by the composition of the N-glycan present at the conserved N-glycosylation site in the Fc region. Hitherto, it has been repeatedly shown that disease-specific antibodies can exhibit skewed glycosylation profiles, which in turn associate with disease prognosis and outcome. Historically, one of the key limitations of glyco-profiling such antibodies is their low serum prevalence and high sample requirement. In order to facilitate Fc glycosylation analysis of low abundant HLA-A2-specific antibodies, an antigen-specific antibody purification approach was developed for reliable, sensitive, and specific capturing of HLA-A2-specific antibodies from reactive sera. This platform was leveraged to support analysis of HLA-A2-specific IgG Fc glycosylation profiles. Compared with the global serum IgG profiles, we observed variable degree of HLA-A2-specific IgGls afucosylation. Altered fucosylation was observed across seropositive patients, whether or not HLA-A2 specificity represented a DSA, a potentially cross-reactive response to the graft, or may have been derived from antigenic exposure(s) unrelated to transplant. Definition as to whether cross-reactive antibodies or antibodies that are specific to distinct donor organ antigens exhibit similar glycoprofiles remains to be determined but has been observed in other disease contexts. This feature was associated with improved FcγRIIIa binding as afucosylation has been described to lead to elevated FcγRIII binding. Additionally, studies have shown that individuals with AMR exhibit higher expression of FCGR3A in NK cells and CD16a-inducible NK cell-selective transcripts compared with individuals without AMR, suggesting that the combination of enriched afucosylated antibodies and elevated FcγRIIIa expression could synergize, leading ADCC to be a key mechanism in graft injury. Hence, we hypothesized that low HLA-A2-specific IgG1 fucosylation could cause elevated DSA cytotoxicity via enhanced ADCC. Indeed, when tested in the context of a subclass-switched recombinant antibody specific for HLA-A2, both subclass and fucosylation had a strong impact on FcγRIIIa affinity and signaling. Specifically, afucosylated mAbs had slower dissociation from the receptor, which could provide sufficient time for the receptors to cross-link and activate downstream signaling pathways and modulate ADCC. This relationship was also observed among polyclonal HLA-A2-specific antibodies purified from patient sera and suggests that afucosylated antibodies to HLA-A2 can exhibit elevated ADCC activity, as shown in other disease settings, and raises the possibility that DSA glycosylation may provide prognostic value in predicting risk of AMR. Indeed, individuals who had AMR exhibited low levels of HLA-A2-specific IgG1 fucosylation compared with the individuals who did not have AMR, suggesting that low HLA-A2 IgG1 fucosylation may be a marker of AMR risk in patients who have had a kidney transplant. Despite the small cohort size, among individuals in whom HLA-A2 represented a DSA, low HLA-A2-specific IgG1 antibody fucosylation was associated with AMR.

TABLE 4
List of sequences.

| SEQ ID NO. | | SEQUENCE |
|---|---|---|
| light chain | 1 | gatgttttgatgacccaaactccactctccctgcctgtcagtcttg |
| | | gagatcaagtctccatctcttgcagatctagtcagagcattgtaca |
| | | tagtaatggaaacacctatttagaatggtacctgcagaaaccaggc |
| | | cagtctccaaagctcctgatctacaaagtttccaaccgattttctg |
| | | gggtcccagacaggttcagtggcagtggatcagggacagatttcac |
| | | actcaagatcagcagagtggaggctgaggatctgggagtttattac |
| | | tgctttcaaggttcacatgttcctcggacgttcggtggaggcacca |
| | | agctggaaatcaaaCGGGCAGATGCTGCACCAACTGTATCCATCTT |
| | | CCCACCATCCAGTGAGCAGTTAACATCTGGAGGTGCCTCAGTCGTG |
| | | TGCTTCTTGAACAACTTCTACCCCAAAGACATCAATGTCAAGTGGA |
| | | AGATTGATGGCAGTGAACGACAAAATGGCGTCCTGAACAGTTGGAC |
| | | TGATCAGGACAGCAAAGACAGCACCTACAGCATGAGCAGCACCCTC |
| | | ACGTTGACCAAGGACGAGTATGAACGACATAACAGCTATACCTGTG |
| | | AGGCCACTCACAAGACATCAACTTCACCCATTGTCAAGAGCTTCAA |
| | | CAGGAATGAGTGT |
| IgG1 heavy chain | 2 | caggtccagctgcagcagtctggacctgagctggtgaagcctgggg |
| | | cctcagtgaagatgtcctgcaaggcttctggctacaccttcacaag |
| | | ctaccatatacagtgggtgaagcagaggcctggacagggacttgag |
| | | tggattggatggatttatcctggagatggtagtactcagtacaatg |
| | | agaagttcaagggcaagaccacactgactgcagacaaatcctccag |
| | | cacagcctacatgttgctcagcagcctgacctctgaggactctgcg |
| | | atctatttctgtgcaagggaggggacctactatgctatggactact |
| | | ggggtcaaggaacctcagtcaccgtctcctcaGCTAAAACAACACC |
| | | CCCATCAGTCTATCCACTGGCCCCTGGGTGTGGAGATACAACTGGT |
| | | TCCTCTGTGACTCTGGGATGCCTGGTCAAGGGCTACTTCCCTGAGT |
| | | CAGTGACTGTGACTTGGAACTCTGGATCCCTGTCCAGCAGTGTGCA |
| | | CACCTTCCCAGCTCTCCTGCAGTCTGGACTCTACACTATGAGCAGC |
| | | TCAGTGACTGTCCCCTCCAGCACCTGGCCAAGTCAGACCGTCACCT |
| | | GCAGCGTTGCTCACCCAGCCAGCAGCACCACGGTGGACAAAAAACT |
| | | TGAGCCCAAATCTTGTGACAAAACTCACACATGCCCACCGTGCCCA |
| | | GCACCTGAACTCCTGGGGGGACCGTCAGTCTTCCTCTTCCCCCCAA |
| | | AACCCAAGGACACCCTCATGATCTCCCGGACCCCTGAGGTCACATG |
| | | CGTGGTGGTGGACGTGAGCCACGAAGACCCTGAGGTCAAGTTCAAC |
| | | TGGTACGTGGACGGCGTGGAGGTGCATAATGCCAAGACAAAGCCGC |
| | | GGGAGGAGCAGTACAACAGCACGTACCGTGTGGTCAGCGTCCTCAC |
| | | CGTCCTGCACCAGGACTGGCTGAATGGCAAGGAGTACAAGTGCAAG |
| | | GTCTCCAACAAAGCCCTCCCAGCCCCCATCGAGAAAACCATCTCCA |
| | | AAGCCAAAGGGCAGCCCCGAGAACCACAGGTGTACACCCTGCCCCC |
| | | ATCCCGGGAGGAGATGACCAAGAACCAGGTCAGCCTGACCTGCCTG |
| | | GTCAAAGGCTTCTATCCCAGCGACATCGCCGTGGAGTGGGAGAGCA |
| | | ATGGGCAGCCGGAGAACAACTACAAGACCACGCCTCCCGTGCTGGA |
| | | CTCCGACGGCTCCTTCTTCCTCTACAGCAAGCTCACCGTGGACAAG |
| | | AGCAGGTGGCAGCAGGGGAACGTCTTCTCATGCTCCGTGATGCATG |
| | | AGGCTCTGCACAACCACTACACGCAGAAGAGCCTCTCCC |
| | | TGTCTCCGGGTAAA |
| IgG2 heavy chain | 3 | caggtccagctgcagcagtctggacctgagctggtgaagcctgggg |
| | | cctcagtgaagatgtcctgcaaggcttctggctacaccttcacaag |
| | | ctaccatatacagtgggtgaagcagaggcctggacagggacttgag |
| | | tggattggatggatttatcctggagatggtagtactcagtacaatg |
| | | agaagttcaagggcaagaccacactgactgcagacaaatcctccag |
| | | cacagcctacatgttgctcagcagcctgacctctgaggactctgcg |
| | | atctatttctgtgcaagggaggggacctactatgctatggactact |
| | | ggggtcaaggaacctcagtcaccgtctcctcaGCTAAAACAACACC |
| | | CCCATCAGTCTATCCACTGGCCCCTGGGTGTGGAGATACAACTGGT |
| | | TCCTCTGTGACTCTGGGATGCCTGGTCAAGGGCTACTTCCCTGAGT |
| | | CAGTGACTGTGACTTGGAACTCTGGATCCCTGTCCAGCAGTGTGCA |
| | | CACCTTCCCAGCTCTCCTGCAGTCTGGACTCTACACTATGAGCAGC |
| | | TCAGTGACTGTCCCCTCCAGCACCTGGCCAAGTCAGACCGTCACCT |
| | | GCAGCGTTGCTCACCCAGCCAGCAGCACCACGGTGGACAAAAAACT |
| | | TGAGCGCAAATGTTGTGTCGAGTGCCCACCGTGCCCAGCACCACCT |
| | | GTGGCAGGACCGTCAGTCTTCCTCTTCCCCCCAAAACCCAAGGACA |
| | | CCCTCATGATCTCCCGGACCCCTGAGGTCACGTGCGTGGTGGTGGA |
| | | CGTGAGCCACGAAGACCCCGAGGTCCAGTTCAACTGGTACGTGGAC |
| | | GGCGTGGAGGTGCATAATGCCAAGACAAAGCCACGGGAGGAGCAGT |
| | | TCAACAGCACGTTCCGTGTGGTCAGCGTCCTCACCGTTGTGCACCA |
| | | GGACTGGCTGAACGGCAAGGAGTACAAGTGCAAGGTCTCCAACAAA |
| | | GGCCTCCCAGCCCCCATCGAGAAAACCATCTCCAAAACCAAAGGGC |
| | | AGCCCCGAGAACCACAGGTGTACACCCTGCCCCCATCCCGGGAGGA |
| | | GATGACCAAGAACCAGGTCAGCCTGACCTGCCTGGTCAAAGGCTTC |
| | | TACCCCAGCGACATCGCCGTGGAGTGGGAGAGCAATGGGCAGCCGG |
| | | AGAACAACTACAAGACCACGCCTCCCATGCTGGACTCCGACGGCTC |
| | | CTTCTTCCTCTACAGCAAGCTCACCGTGGACAAGAGCAGGTGGCAG |
| | | CAGGGGAACGTCTTCTCATGCTCCGTGATGCATGAGGCTCTGCACA |
| | | ACCACTACACGCAGAAGAGCCTCTCCCTGTCTCCGGGTAAA |
| IgG3 heavy chain | 4 | caggtccagctgcagcagtctggacctgagctggtgaagcctgggg |
| | | cctcagtgaagatgtcctgcaaggcttctggctacaccttcacaag |
| | | ctaccatatacagtgggtgaagcagaggcctggacagggacttgag |
| | | tggattggatggatttatcctggagatggtagtactcagtacaatg |
| | | agaagttcaagggcaagaccacactgactgcagacaaatcctccag |
| | | cacagcctacatgttgctcagcagcctgacctctgaggactctgcg |
| | | atctatttctgtgcaagggaggggacctactatgctatggactact |
| | | ggggtcaaggaacctcagtcaccgtctcctcaGCTAAAACAACACC |
| | | CCCATCAGTCTATCCACTGGCCCCTGGGTGTGGAGATACAACTGGT |
| | | TCCTCTGTGACTCTGGGATGCCTGGTCAAGGGCTACTTCCCTGAGT |
| | | CAGTGACTGTGACTTGGAACTCTGGATCCCTGTCCAGCAGTGTGCA |
| | | CACCTTCCCAGCTCTCCTGCAGTCTGGACTCTACACTATGAGCAGC |
| | | TCAGTGACTGTCCCCTCCAGCACCTGGCCAAGTCAGACCGTCACCT |
| | | GCAGCGTTGCTCACCCAGCCAGCAGCACCACGGTGGACAAAAAACT |
| | | TGAATTGAAGACACCTCTCGGCGACACTACTCACACATGCCCAAGA |
| | | TGCCCAGAGCCTAAGTCCTGCGACACCCCTCCTCCCTGTCCTAGAT |
| | | GCCCTGAGCCAAAGTCTTGCGATACGCCTCCACCCTGCCCTCGGTG |
| | | TCCTGAGCCTAAATCATGCGATACCCCACCACCATGTCCTCGCTGC |
| | | CCCGCACCTGAACTCCTGGGAGGACCGTCAGTCTTCCTCTTCCCCC |
| | | CAAAACCCAAGGATACCCTTATGATTTCCCGGACCCCTGAGGTCAC |
| | | GTGCGTGGTGGTGGACGTGAGCCACGAAGACCCCGAGGTCCAGTTC |
| | | AAGTGGTACGTGGACGGCGTGGAGGTGCATAATGCCAAGACAAAGC |
| | | CGCGGGAGGAGCAGTACAACAGCACGTTCCGTGTGGTCAGCGTCCT |
| | | CACCGTCCTGCACCAGGACTGGCTGAACGGCAAGGAGTACAAGTGC |
| | | AAGGTCTCCAACAAAGCCCTCCCAGCCCCCATCGAGAAAACCATCT |
| | | CCAAAACCAAAGGACAGCCCCGAGAACCACAGGTGTACACCCTGCC |
| | | CCCATCCCGGGAGGAGATGACCAAGAACCAGGTCAGCCTGACCTGC |
| | | CTGGTCAAAGGCTTCTACCCCAGCGACATCGCCGTGGAGTGGGAGA |
| | | GCAGCGGGCAGCCGGAGAACAACTACAACACCACGCCTCCCATGCT |
| | | GGACTCCGACGGCTCCTTCTTCCTCTACAGCAAGCTCACCGTGGAC |
| | | AAGAGCAGGTGGCAGCAGGGGAACATCTTCTCATGCTCCGTGATGC |
| | | ATGAGGCTCTGCACAACCGCTTCACGCAGAAGAGCCTCTCCCTGTC |
| | | TCCGGGTAAA |
| IgG4 heavy chain | 5 | caggtccagctgcagcagtctggacctgagctggtgaagcctgggg |
| | | cctcagtgaagatgtcctgcaaggcttctggctacaccttcacaag |
| | | ctaccatatacagtgggtgaagcagaggcctggacagggacttgag |
| | | tggattggatggatttatcctggagatggtagtactcagtacaatg |
| | | agaagttcaagggcaagaccacactgactgcagacaaatcctccag |
| | | cacagcctacatgttgctcagcagcctgacctctgaggactctgcg |
| | | atctatttctgtgcaagggaggggacctactatgctatggactact |
| | | ggggtcaaggaacctcagtcaccgtctcctcaGCTAAAACAACACC |
| | | CCCATCAGTCTATCCACTGGCCCCTGGGTGTGGAGATACAACTGGT |
| | | TCCTCTGTGACTCTGGGATGCCTGGTCAAGGGCTACTTCCCTGAGT |
| | | CAGTGACTGTGACTTGGAACTCTGGATCCCTGTCCAGCAGTGTGCA |
| | | CACCTTCCCAGCTCTCCTGCAGTCTGGACTCTACACTATGAGCAGC |
| | | TCAGTGACTGTCCCCTCCAGCACCTGGCCAAGTCAGACCGTCACCT |
| | | GCAGCGTTGCTCACCCAGCCAGCAGCACCACGGTGGACAAAAAACT |
| | | TGAGTCCAAATATGGTCCCCCATGCCCATCATGCCCAGCACCTGAG |
| | | TTCCTGGGGGGACCATCAGTCTTCCTGTTCCCCCCAAAACCCAAGG |
| | | ACACTCTCATGATCTCCCGGACCCCTGAGGTCACGTGCGTGGTGGT |
| | | GGACGTGAGCCAGGAAGACCCCGAGGTCCAGTTCAACTGGTACGTG |
| | | GATGGCGTGGAGGTGCATAATGCCAAGACAAAGCCGCGGGAGGAGC |
| | | AGTTCAACAGCACGTACCGTGTGGTCAGCGTCCTCACCGTCCTGCA |
| | | CCAGGACTGGCTGAACGGCAAGGAGTACAAGTGCAAGGTCTCCAAC |
| | | AAAGGCCTCCCGTCCTCCATCGAGAAAACCATCTCCAAAGCCAAAG |
| | | GGCAGCCCCGAGAGCCACAGGTGTACACCCTGCCCCCATCCCAGGA |
| | | GGAGATGACCAAGAACCAGGTCAGCCTGACCTGCCTGGTCAAAGGC |
| | | TTCTACCCCAGCGACATCGCCGTGGAGTGGGAGAGCAATGGGCAGC |
| | | CGGAGAACAACTACAAGACCACGCCTCCCGTGCTGGACTCCGACGG |
| | | CTCCTTCTTCCTCTACAGCAGGCTCACCGTGGACAAGAGCAGGTGG |
| | | CAGGAGGGGAATGTCTTCTCATGCTCCGTGATGCATGAGGCTCTGC |
| | | ACAACCACTACACACAGAAGAGCCTCTCCCTGTCTCCGGGTAAA |
| EBV membrane protein | 6 | CLGGLLTMV |
| EBV, mRNA export | 7 | GLCTLVAML |
| factor ICP27 | | |
| CMV, pp50 | 8 | VTEHDTLLY |
| Influenza A virus, | 9 | CTELKLSDY |
| Nucleoprotein | | |

1. A method for assessing if a candidate for or a recipient of a solid organ allograft is at risk of developing antibody-mediated rejection (AMR), the method comprising:
determining or having determined a feature of a donor-specific antibody (DSA) in a sample obtained from the candidate or recipient; and
identifying the candidate or the recipient as having a high risk of AMR based on the DSA having a low level of fucosylation and/or exhibiting slow dissociation from FcγRIIIa, enhanced FcγRIIIa signaling, and/or ADCC activity

2. The method of claim 1, further comprising (i) allocating a solid organ allograft for the candidate; (ii) selecting the candidate for transplant surgery; and/or (iii) selecting the candidate for treatment with an immunosuppressive therapy based on the feature(s) of the DSA, preferably wherein the immunosuppressive therapy is an immunosuppressive induction therapy.

3. (canceled)

4. The method of claim 1, further comprising selecting the recipient for treatment with an immunosuppressive therapy based on the feature(s) of the DSA, preferably wherein the immunosuppressive therapy is an immunosuppressive maintenance therapy.

5. (canceled)

6. The method of claim 1, further comprising treating the candidate or the recipient with an immunosuppressive therapy if the candidate or recipient has a high risk of AMR.

7. The method of claim 1, wherein the feature of the DSA is selected from the group consisting of IgG subclass, Fc domain glycosylation, FcγR binding properties, and combinations thereof.

8. The method of claim 1, wherein the feature of the DSA is an Fc feature selected from the group consisting of Fc domain glycosylation and FcγR binding properties.

9. The method of claim 8, wherein the DSA is HLA-A2-specific IgG1.

10. A method for identifying and treating a renal allograft candidate or a renal allograft recipient at risk of developing antibody-mediated rejection (AMR), the method comprising:
(a) determining or having determined a feature of a donor-specific antibody (DSA) in a sample obtained from the candidate or recipient;
(b) quantitatively or qualitatively comparing the feature of the DSA with a reference value;
(c) identifying the renal allograft candidate or recipient as being at risk for allograft rejection based the comparison in step (b), and
(d) providing a therapeutic intervention to the candidate or recipient identified in step (c) as being at risk of allograft rejection

11. The method of claim 10, wherein the reference value is obtained from a corresponding feature previously determined from samples obtained from renal allograft recipients who did not have clinically defined AMR or a group of otherwise healthy individuals.

12. The method of claim 10, wherein the therapeutic intervention comprises administration of one or more immunosuppressants, induction immunosuppressive therapy, or maintenance immunosuppressive therapy.

13. (canceled)

14. (canceled)

15. The method of claim 10, wherein the feature of the DSA is selected from the group consisting of IgG subclass, Fc domain glycosylation, FcγR binding properties, and combinations thereof.

16. The method of claim 10, wherein the feature of the DSA is an Fc feature selected from the group consisting of Fc domain glycosylation and FcγR binding properties.

17. The method of claim 16, wherein the DSA is HLA-A2-specific IgG1.

18. An in vitro method for determining the likelihood of occurrence of antibody-mediated rejection (AMR) against a renal allograft in a transplant candidate or recipient, the method comprising:
(a) obtaining a sample containing donor-specific antibodies (DSAs) from the candidate or recipient;
(b) affinity purifying DSAs from the sample using a donor-specific antigen attached to a solid support;
(c) enzymatically digesting the affinity purified DSAs to obtain Fc glycopeptides;
(d) determining glycosylation profiles of the Fc glycopeptides obtained in step (c);
(e) comparing the Fc glycosylation profile obtained in step (d) with a reference value; and
(f) determining the likelihood of occurrence of AMR against a renal allograft in the candidate or recipient based on the comparison of step (e).

19. The method of claim 18, wherein the sample is a serum sample.

20. The method of claim 18, wherein the solid support is a magnetic, streptavidin coated bead.

21. The method of claim 18, wherein the enzymatic digestion comprises tryptic digestion.

22. The method of claim 18, wherein the reference value is obtained from an Fc glycosylation profile previously determined from samples obtained from renal allograft recipients who did not have clinically defined AMR or a group of otherwise healthy individuals.

23. The method of claim 18, wherein the glycosylation profile is a fucosylation profile.

24. The method of claim 23, wherein a low level of DSA fucosylation indicates a high likelihood of occurrence AMR.

25. The method of claim 18, wherein the DSA is HLA-A2-specific IgG1 and/or the donor-specific antigen is HLA-A2.

26. A method for treating antibody-mediated rejection (AMR) in a candidate for or a recipient of a solid organ allograft, particularly a renal allograft, the method comprising:
(a) determining the likelihood of occurrence of AMR by
(1) determining or having determined a feature of a donor-specific antibody (DSA) in a sample obtained from the candidate or recipient;
(2) quantitatively or qualitatively comparing the feature of the DSA determined in step (a)(1) with a reference value;
(3) determining the likelihood of occurrence of AMR based on the comparison of step (a)(2);
(b) selecting the candidate or recipient when the candidate or recipient has been determined as being likely to develop AMR at step (a); and
(c) treating the candidate or recipient selected at step (b) with an immunosuppressive therapy.

27. The method of claim 26, wherein the sample is a serum sample.

28. The method of claim 26, wherein the reference value is obtained from a corresponding feature previously determined from samples obtained from renal allograft recipients who did not have clinically defined AMR or a group of otherwise healthy individuals.

29. The method of claim 26, wherein the immunosuppressive therapy comprises induction immunosuppressive therapy.

30. The method of claim 26, wherein the immunosuppressive therapy comprises maintenance immunosuppressive therapy.

31. The method of claim 26, wherein the feature of the DSA is selected from the group consisting of IgG subclass, Fc domain glycosylation, FcγR binding properties, and combinations thereof.

32. The method of claim 31, wherein the DSA is HLA-A2-specific IgG1.


SEQUENCE LISTING (9 sequences)

SEQ ID NO: 1 - DNA, 657 bp, synthetic construct
  source 1..657: mol_type = other DNA
gatgttttgatgacccaaactccactctccctgcctgtcagtcttggagatcaagtctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttcctcggacgttcggtggaggcaccaagctggaaatcaaacgggcagatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 2 - DNA, 1341 bp, synthetic construct
  source 1..1341: mol_type = other DNA
caggtccagctgcagcagtctggacctgagctggtgaagcctggggcctcagtgaagatgtcctgcaaggcttctggctacaccttcacaagctaccatatacagtgggtgaagcagaggcctggacagggacttgagtggattggatggatttatcctggagatggtagtactcagtacaatgagaagttcaagggcaagaccacactgactgcagacaaatcctccagcacagcctacatgttgctcagcagcctgacctctgaggactctgcgatctatttctgtgcaagggaggggacctactatgctatggactactggggtcaaggaacctcagtcaccgtctcctcagctaaaacaacacccccatcagtctatccactggcccctgggtgtggagatacaactggttcctctgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 3 - DNA, 1329 bp, synthetic construct
  source 1..1329: mol_type = other DNA
caggtccagctgcagcagtctggacctgagctggtgaagcctggggcctcagtgaagatgtcctgcaaggcttctggctacaccttcacaagctaccatatacagtgggtgaagcagaggcctggacagggacttgagtggattggatggatttatcctggagatggtagtactcagtacaatgagaagttcaagggcaagaccacactgactgcagacaaatcctccagcacagcctacatgttgctcagcagcctgacctctgaggactctgcgatctatttctgtgcaagggaggggacctactatgctatggactactggggtcaaggaacctcagtcaccgtctcctcagctaaaacaacacccccatcagtctatccactggcccctgggtgtggagatacaactggttcctctgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 4 - DNA, 1482 bp, synthetic construct
  source 1..1482: mol_type = other DNA
caggtccagctgcagcagtctggacctgagctggtgaagcctggggcctcagtgaagatgtcctgcaaggcttctggctacaccttcacaagctaccatatacagtgggtgaagcagaggcctggacagggacttgagtggattggatggatttatcctggagatggtagtactcagtacaatgagaagttcaagggcaagaccacactgactgcagacaaatcctccagcacagcctacatgttgctcagcagcctgacctctgaggactctgcgatctatttctgtgcaagggaggggacctactatgctatggactactggggtcaaggaacctcagtcaccgtctcctcagctaaaacaacacccccatcagtctatccactggcccctgggtgtggagatacaactggttcctctgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgaattgaagacacctctcggcgacactactcacacatgcccaagatgcccagagcctaagtcctgcgacacccctcctccctgtcctagatgccctgagccaaagtcttgcgatacgcctccaccctgccctcggtgtcctgagcctaaatcatgcgataccccaccaccatgtcctcgctgccccgcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaa

SEQ ID NO: 5 - DNA, 1332 bp, synthetic construct
  source 1..1332: mol_type = other DNA
caggtccagctgcagcagtctggacctgagctggtgaagcctggggcctcagtgaagatgtcctgcaaggcttctggctacaccttcacaagctaccatatacagtgggtgaagcagaggcctggacagggacttgagtggattggatggatttatcctggagatggtagtactcagtacaatgagaagttcaagggcaagaccacactgactgcagacaaatcctccagcacagcctacatgttgctcagcagcctgacctctgaggactctgcgatctatttctgtgcaagggaggggacctactatgctatggactactggggtcaaggaacctcagtcaccgtctcctcagctaaaacaacacccccatcagtctatccactggcccctgggtgtggagatacaactggttcctctgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctccgggtaaa

SEQ ID NO: 6 - protein, 9 aa, synthetic construct
CLGGLLTMV

SEQ ID NO: 7 - protein, 9 aa, synthetic construct
GLCTLVAML

SEQ ID NO: 8 - protein, 9 aa, synthetic construct
VTEHDTLLY

SEQ ID NO: 9 - protein, 9 aa, synthetic construct
CTELKLSDY